(19) United States
(12) Patent Application Publication
(10) Pub. No.: US 2022/0059184 A1
(43) Pub. Date: Feb. 24, 2022

(54) METHODS FOR IDENTIFYING EPITOPES AND PARATOPES

(71) Applicant: VISTERRA, INC., Waltham, MA (US)

(72) Inventors: Andrew M. Wollacott, Milton, MA (US); Luke Robinson, Quincy, MA (US); Boopathy Ramakrishnan, Braintree, MA (US); Hamid Tissire, Millis, MA (US); Karthik Viswanathan, Acton, MA (US); Zachary Shriver, Winchester, MA (US); Gregory Babcock, Marlborough, MA (US)

(21) Appl. No.: 17/417,597

(22) PCT Filed: Dec. 23, 2019
(86) PCT No.: PCT/US2019/068346

§ 371 Date: Jun. 23, 2021

Related U.S. Application Data
(60) Provisional application No. 62/784,617, filed on Dec. 24, 2018.

(51) Int. Cl. G16B 15/30; G16B 35/20; C07K 16/28

(52) U.S. Cl. CPC G16B 15/30; C07K 2317/34; C07K 16/2875; G16B 35/20

(57) ABSTRACT

Disclosed are methods of identifying an epitope on a target polypeptide and methods of identifying a paratope on an antibody.

### CROSS REFERENCE TO RELATED APPLICATIONS

This application claims the benefit of U.S. Provisional Application No. 62/784,617, filed Dec. 24, 2018. The contents of the aforesaid application are hereby incorporated by reference in its entirety.

### BACKGROUND

Antibodies bind target antigens with high specificity and affinity. Molecularly, binding is facilitated by the set of amino acids in the antibody (paratope) and the antigen (epitope) which contribute to energetically favorable interactions for binding to occur. Determining the structural features governing antibody-antigen interactions is important for understanding an antibody's mechanism of action and as a reference to aid antibody engineering efforts. X-ray co-crystallography is a leading method to determine the structure of antibody-antigen complexes, detailing both the structural paratope and epitope with high resolution. However, achievement of high resolution co-crystal structures has considerable resource, throughput, and specialized technical expertise requirements. Other methods to characterize paratopes and epitopes provide greater throughput and experimental accessibility but typically come with a tradeoff of resolution. Epitope binning by competition binding or epitope characterization by alanine scanning each provide greater speed and throughput than crystallography but cannot provide the molecular detail nor the comprehensiveness of characterization as in crystallography. Thus, there exists a need in the art for improved methods of identifying epitope and paratope regions between an antibody and its recognized antigen.

### SUMMARY

In an aspect, the disclosure features a method of identifying an epitope on a target polypeptide (e.g., a target polypeptide described herein), the method comprising:

(a) binding an antibody molecule (e.g., an antibody molecule described herein) to a plurality of variants of the target polypeptide;

(b) obtaining (e.g., enriching) a plurality of variants exhibiting altered (e.g., reduced) binding to the antibody molecule;

(c) determining (e.g., calculating) an enrichment score for each of the plurality of the obtained (e.g., enriched) variants;

(d) generating an antibody molecule-target polypeptide docking model, wherein the antibody molecule-target polypeptide docking model is constrained according to the enrichment scores; and

(e) identifying a site on the target polypeptide that is capable of being bound by the antibody molecule based on the antibody molecule-target polypeptide docking model;

thereby identifying an epitope on a target polypeptide.

In an embodiment, the altered binding comprises altered binding affinity, e.g., reduced binding affinity.

In an embodiment, step (a) comprises binding the antibody molecule to a library displaying a plurality of variants of the target polypeptide. In an embodiment, step (a) comprises binding the antibody molecule to a library comprising a plurality of cells expressing (e.g., displaying) a plurality of variants of the target polypeptide. In an embodiment, each of the plurality of cells expresses about one distinct variant of the target polypeptide. In an embodiment, the cell is a eukaryotic cell, e.g., a yeast cell.

In an embodiment, the plurality of variants comprise mutations on one or more surface residues of the target polypeptide. In an embodiment, the plurality of variants comprise distinct mutations of a selected surface residue of the target polypeptide. In an embodiment, the plurality of variants comprise distinct mutations of each of a plurality of selected surface residues of the target polypeptide.

In an embodiment, the plurality of variants comprise single amino acid substitutions, relative to a wild-type amino acid sequence of the target polypeptide. In an embodiment, each of the plurality of variants comprises a single amino acid substitution relative to a wild-type amino acid sequence of the target polypeptide. In an embodiment, the single amino acid substitution occurs at a surface residue of the target polypeptide.

In an embodiment, the altered (e.g., reduced) binding comprises an alteration (e.g., a reduction) of binding detected for the variant and the antibody molecule, relative to the binding detected for a wild-type target polypeptide and the antibody.

In an embodiment, step (b) comprises obtaining (e.g., enriching) variants exhibiting less than about 80% (e.g., less than about 0.01%, 0.1%, 1%, 2%, 3%, 4%, 5%, 6%, 7%, 8%, 9%, 10%, 20%, 30%, 40%, 50%, 60%, 70%, or 80%) of the binding to the antibody molecule exhibited by a wild-type target polypeptide. In an embodiment, the reduced binding is at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by the wild-type target polypeptide.

In an embodiment, step (b) comprises obtaining (e.g., enriching) cells exhibiting less than about 80% (e.g., less than about 0.01%, 0.1%, 1%, 2%, 3%, 4%, 5%, 6%, 7%, 8%, 9%, 10%, 20%, 30%, 40%, 50%, 60%, 70%, or 80%) of the binding to the antibody molecule exhibited by a cell comprising a wild-type target polypeptide. In an embodiment, the reduced binding is at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by a cell comprising the wild-type target polypeptide.

In an embodiment, step (b) comprises performing one or more, e.g., two, three, four, five, six, seven, eight, nine, ten, or more, enrichments for variants exhibiting reduced binding to the antibody molecule.

In an embodiment, the method further comprises, e.g., prior to step (c), identifying the variants exhibiting altered (e.g., reduced) binding to the antibody molecule, e.g., by sequencing the genes encoding the variants, e.g., by next-generation sequencing.

In an embodiment, step (c) comprises determining the frequency of occurrence for each of the plurality of the obtained (e.g., enriched) variants. In an embodiment, step (c) further comprises aggregating the frequency of occurrence of each variant comprising a distinct mutation at a particular residue and/or weighting (e.g., heavily weighting) variants with higher frequencies of occurrence.

In an embodiment, the enrichment score is specific to a single residue of the amino acid sequence of the target polypeptide. In an embodiment, each enrichment score is specific to a different single residue of the amino acid sequence of the target polypeptide.

In an embodiment, the method further comprises repeating steps (a)-(c) at least once (e.g., once, twice, three times, four times, five times, six times, seven times, eight times, nine times, ten times, or more) with replicates of the plurality of the variants of the target polypeptide, and wherein step (c) further comprises omitting one or more promiscuous mutations, e.g., mutations for which more than 50% of replicates had an enrichment score of greater than 30% and for which more than 75% of replicates had an enrichment score greater than 15%.

In an embodiment, the antibody molecule-target polypeptide docking model is constrained by adding one or more attractive constraints, optionally, wherein the attractive constraint is for a residue having an enrichment score greater than a first preselected value. In an embodiment, the first preselected value is between 20% and 40%, e.g., between 25% and 35%, e.g., about 25%, about 30%, or about 35%. In an embodiment, the attractive constraint comprises a linearly scaled bonus based on the enrichment score.

In an embodiment, the antibody molecule-target polypeptide docking model is constrained by adding a repulsive constraint for a residue having an enrichment score less than a second preselected value. In an embodiment, the second preselected value is between 5% and 20%, e.g., between 10% and 15%, e.g., about 10%, about 12.5%, or about 15%.

In an embodiment, step (d) comprises generating a docked pose between a model of the antibody molecule and a model of the target polypeptide. In an embodiment, step (d) comprises generating a plurality of docked poses between a model of the antibody molecule and a model of the target polypeptide.

In an embodiment, step (d) further comprises scoring the plurality of docked poses according to a docking algorithm, e.g., SnugDock. In an embodiment, step (d) further comprises selecting a subset of the plurality of docked poses having the highest scores, e.g., the highest scoring 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000 or more docked poses. In an embodiment, step (d) further comprises generating an ensemble docked pose using the selected subset of the plurality of docked poses, and setting the model of the antibody molecule and the model of the target polypeptide in accordance with the ensemble docked pose.

In an embodiment, the model of the antibody molecule comprises an ensemble antibody homology model derived from a plurality of homology models of the antibody.

In an embodiment, step (d) further comprises removing an antibody molecule-target polypeptide docketing model that exhibits a mode of engagement atypical for a known antibody-antigen complex, e.g., according to a structural filter derived from antibody-antigen crystal structure.

In an embodiment, step (d) comprises generating a plurality of antibody molecule-target polypeptide models.

In an embodiment, step (e) comprises identifying a plurality of sites on the target polypeptide that is capable of being bound by the antibody molecule.

In an embodiment, the site comprises or consists of one or more non-consecutive regions on the target polypeptide. In an embodiment, the site comprises or consists of a consecutive region on the target polypeptide.

In another aspect, the disclosure features a method of identifying an epitope on a target polypeptide (e.g., a target polypeptide described herein), the method comprising:

(a) generating an antibody-target polypeptide docking model, wherein the antibody-target polypeptide docking model is constrained according to a plurality of enrichment scores determined by a method comprising:
(i) binding an antibody molecule (e.g., an antibody molecule described herein) to a plurality of variants of the target polypeptide,
(ii) obtaining (e.g., enriching) a plurality of variants exhibiting altered (e.g., reduced) binding to the antibody molecule, and
(iii) determining (e.g., calculating) enrichment scores for each of the plurality of the enriched variants; and

(b) identifying a site on the target polypeptide that is capable of being bound by the antibody molecule based on the antibody-target polypeptide docking model;

thereby identifying an epitope on a target polypeptide.

In an embodiment, the altered binding comprises altered binding affinity, e.g., reduced binding affinity.

In an embodiment, step (a)(i) comprises binding the antibody molecule to a library displaying a plurality of variants of the target polypeptide. In an embodiment, step (a)(i) comprises binding the antibody molecule to a library comprising a plurality of cells expressing (e.g., displaying) a plurality of variants of the target polypeptide. In an embodiment, each of the plurality of cells expresses about one distinct variant of the target polypeptide. In an embodiment, the cell is a eukaryotic cell, e.g., a yeast cell.

In an embodiment, the plurality of variants comprise mutations on one or more surface residues of the target polypeptide. In an embodiment, the plurality of variants comprise distinct mutations of a selected surface residue of the target polypeptide. In an embodiment, the plurality of variants comprise distinct mutations of each of a plurality of selected surface residues of the target polypeptide.

In an embodiment, the plurality of variants comprise single amino acid substitutions, relative to a wild-type amino acid sequence of the target polypeptide. In an embodiment, each of the plurality of variants comprises a single amino acid substitution relative to a wild-type amino acid sequence of the target polypeptide. In an embodiment, the single amino acid substitution occurs at a surface residue of the target polypeptide.

In an embodiment, the altered (e.g., reduced) binding comprises an alteration (e.g., a reduction) of binding detected for the variant and the antibody molecule, relative to the binding detected for a wild-type target polypeptide and the antibody.

In an embodiment, step (a)(ii) comprises obtaining (e.g., enriching) variants exhibiting less than about 80% (e.g., less than about 0.01%, 0.1%, 1%, 2%, 3%, 4%, 5%, 6%, 7%, 8%, 9%, 10%, 20%, 30%, 40%, 50%, 60%, 70%, or 80%) of the binding to the antibody molecule exhibited by a wild-type target polypeptide. In an embodiment, the reduced binding is at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by the wild-type target polypeptide.

In an embodiment, step (a)(ii) comprises obtaining (e.g., enriching) cells exhibiting less than about 80% (e.g., less than about 0.01%, 0.1%, 1%, 2%, 3%, 4%, 5%, 6%, 7%, 8%, 9%, 10%, 20%, 30%, 40%, 50%, 60%, 70%, or 80%) of the binding to the antibody molecule exhibited by a cell comprising a wild-type target polypeptide. In an embodiment, the reduced binding is at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by a cell comprising the wild-type target polypeptide.

In an embodiment, step (a)(ii) comprises performing one or more, e.g., two, three, four, five, six, seven, eight, nine, ten, or more, enrichments for variants exhibiting reduced binding to the antibody molecule.

In an embodiment, the method further comprises, e.g., prior to step (a)(iii), identifying the variants exhibiting altered (e.g., reduced) binding to the antibody molecule, e.g., by sequencing the genes encoding the variants, e.g., by next-generation sequencing.

In an embodiment, step (a)(iii) comprises determining the frequency of occurrence for each of the plurality of the obtained (e.g., enriched) variants. In an embodiment, step (a)(iii) further comprises aggregating the frequency of occurrence of each variant comprising a distinct mutation at a particular residue and/or weighting (e.g., heavily weighting) variants with higher frequencies of occurrence.

In an embodiment, the enrichment score is specific to a single residue of the amino acid sequence of the target polypeptide. In an embodiment, each enrichment score is specific to a different single residue of the amino acid sequence of the target polypeptide.

In an embodiment, the method further comprises repeating steps (a)(i)-(a)(iii) at least once (e.g., once, twice, three times, four times, five times, six times, seven times, eight times, nine times, ten times, or more) with replicates of the plurality of the variants of the target polypeptide, and wherein step (a)(iii) further comprises omitting one or more promiscuous mutations, e.g., mutations for which more than 50% of replicates had an enrichment score of greater than 30% and for which more than 75% of replicates had an enrichment score greater than 15%.

In an embodiment, the antibody molecule-target polypeptide docking model is constrained by adding one or more attractive constraints, optionally, wherein the attractive constraint is for a residue having an enrichment score greater than a first preselected value. In an embodiment, the first preselected value is between 20% and 40%, e.g., between 25% and 35%, e.g., about 25%, about 30%, or about 35%. In an embodiment, the attractive constraint comprises a linearly scaled bonus based on the enrichment score.

In an embodiment, the antibody molecule-target polypeptide docking model is constrained by adding a repulsive constraint for a residue having an enrichment score less than a second preselected value. In an embodiment, the second preselected value is between 5% and 20%, e.g., between 10% and 15%, e.g., about 10%, about 12.5%, or about 15%.

In an embodiment, step (a) comprises generating a docked pose between a model of the antibody molecule and a model of the target polypeptide. In an embodiment, step (a) comprises generating a plurality of docked poses between a model of the antibody molecule and a model of the target polypeptide.

In an embodiment, step (a) further comprises scoring the plurality of docked poses according to a docking algorithm, e.g., SnugDock. In an embodiment, step (a) further comprises selecting a subset of the plurality of docked poses having the highest scores, e.g., the highest scoring 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000 or more docked poses. In an embodiment, step (a) further comprises generating an ensemble docked pose using the selected subset of the plurality of docked poses, and setting the model of the antibody molecule and the model of the target polypeptide in accordance with the ensemble docked pose.

In an embodiment, the model of the antibody molecule comprises an ensemble antibody homology model derived from a plurality of homology models of the antibody.

In an embodiment, step (a) further comprises removing an antibody molecule-target polypeptide docketing model that exhibits a mode of engagement atypical for a known antibody-antigen complex, e.g., according to a structural filter derived from antibody-antigen crystal structure.

In an embodiment, step (a) comprises generating a plurality of antibody molecule-target polypeptide models.

In an embodiment, step (b) comprises identifying a plurality of sites on the target polypeptide that is capable of being bound by the antibody molecule.

In an embodiment, the site comprises or consists of one or more non-consecutive regions on the target polypeptide. In an embodiment, the site comprises or consists of a consecutive region on the target polypeptide.

In yet another aspect, the disclosure features a method of identifying a paratope on an antibody molecule, the method comprising:

(a) binding the antibody molecule to a plurality of variants of the target polypeptide;

(b) obtaining (e.g., enriching) a plurality of variants exhibiting reduced binding to the antibody molecule;

(c) determining (e.g., calculating) enrichment scores for each of the plurality of the enriched variants;

(d) generating an antibody molecule-target polypeptide docking model, wherein the antibody-target polypeptide docking model is constrained according to the enrichment scores; and

(e) identifying one or more sites on the antibody molecule that is capable of being bound by the target polypeptide based on the antibody-target polypeptide docking model;

thereby identifying a paratope on an antibody molecule.

In an embodiment, the altered binding comprises altered binding affinity, e.g., reduced binding affinity.

In an embodiment, step (a) comprises binding the antibody molecule to a library displaying a plurality of variants of the target polypeptide. In an embodiment, step (a) comprises binding the antibody molecule to a library comprising a plurality of cells expressing (e.g., displaying) a plurality of variants of the target polypeptide. In an embodiment, each of the plurality of cells expresses about one distinct variant of the target polypeptide. In an embodiment, the cell is a eukaryotic cell, e.g., a yeast cell.

In an embodiment, the plurality of variants comprise mutations on one or more surface residues of the target polypeptide. In an embodiment, the plurality of variants comprise distinct mutations of a selected surface residue of the target polypeptide. In an embodiment, the plurality of variants comprise distinct mutations of each of a plurality of selected surface residues of the target polypeptide.

In an embodiment, the plurality of variants comprise single amino acid substitutions, relative to a wild-type amino acid sequence of the target polypeptide. In an embodiment, each of the plurality of variants comprises a single amino acid substitution relative to a wild-type amino acid sequence of the target polypeptide. In an embodiment, the single amino acid substitution occurs at a surface residue of the target polypeptide.

In an embodiment, the altered (e.g., reduced) binding comprises an alteration (e.g., a reduction) of binding detected for the variant and the antibody molecule, relative to the binding detected for a wild-type target polypeptide and the antibody.

In an embodiment, step (b) comprises obtaining (e.g., enriching) variants exhibiting less than about 80% (e.g., less than about 0.01%, 0.1%, 1%, 2%, 3%, 4%, 5%, 6%, 7%, 8%, 9%, 10%, 20%, 30%, 40%, 50%, 60%, 70%, or 80%) of the binding to the antibody molecule exhibited by a wild-type target polypeptide. In an embodiment, the reduced binding is at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by the wild-type target polypeptide.

In an embodiment, step (b) comprises obtaining (e.g., enriching) cells exhibiting less than about 80% (e.g., less than about 0.01%, 0.1%, 1%, 2%, 3%, 4%, 5%, 6%, 7%, 8%, 9%, 10%, 20%, 30%, 40%, 50%, 60%, 70%, or 80%) of the binding to the antibody molecule exhibited by a cell comprising a wild-type target polypeptide. In an embodiment, the reduced binding is at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by a cell comprising the wild-type target polypeptide.

In an embodiment, step (b) comprises performing one or more, e.g., two, three, four, five, six, seven, eight, nine, ten, or more, enrichments for variants exhibiting reduced binding to the antibody molecule.

In an embodiment, the method further comprises, e.g., prior to step (c), identifying the variants exhibiting altered (e.g., reduced) binding to the antibody molecule, e.g., by sequencing the genes encoding the variants, e.g., by next-generation sequencing.

In an embodiment, step (c) comprises determining the frequency of occurrence for each of the plurality of the obtained (e.g., enriched) variants. In an embodiment, step (c) further comprises aggregating the frequency of occurrence of each variant comprising a distinct mutation at a particular residue and/or weighting (e.g., heavily weighting) variants with higher frequencies of occurrence.

In an embodiment, the enrichment score is specific to a single residue of the amino acid sequence of the target polypeptide. In an embodiment, each enrichment score is specific to a different single residue of the amino acid sequence of the target polypeptide.

In an embodiment, the method further comprises repeating steps (a)-(c) at least once (e.g., once, twice, three times, four times, five times, six times, seven times, eight times, nine times, ten times, or more) with replicates of the plurality of the variants of the target polypeptide, and wherein step (c) further comprises omitting one or more promiscuous mutations, e.g., mutations for which more than 50% of replicates had an enrichment score of greater than 30% and for which more than 75% of replicates had an enrichment score greater than 15%.

In an embodiment, the antibody molecule-target polypeptide docking model is constrained by adding one or more attractive constraints, optionally, wherein the attractive constraint is for a residue having an enrichment score greater than a first preselected value. In an embodiment, the first preselected value is between 20% and 40%, e.g., between 25% and 35%, e.g., about 25%, about 30%, or about 35%. In an embodiment, the attractive constraint comprises a linearly scaled bonus based on the enrichment score.

In an embodiment, the antibody molecule-target polypeptide docking model is constrained by adding a repulsive constraint for a residue having an enrichment score less than a second preselected value. In an embodiment, the second preselected value is between 5% and 20%, e.g., between 10% and 15%, e.g., about 10%, about 12.5%, or about 15%.

In an embodiment, step (d) comprises generating a docked pose between a model of the antibody molecule and a model of the target polypeptide. In an embodiment, step (d) comprises generating a plurality of docked poses between a model of the antibody molecule and a model of the target polypeptide.

In an embodiment, step (d) further comprises scoring the plurality of docked poses according to a docking algorithm, e.g., SnugDock. In an embodiment, step (d) further comprises selecting a subset of the plurality of docked poses having the highest scores, e.g., the highest scoring 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000 or more docked poses. In an embodiment, step (d) further comprises generating an ensemble docked pose using the selected subset of the plurality of docked poses, and setting the model of the antibody molecule and the model of the target polypeptide in accordance with the ensemble docked pose.

In an embodiment, the model of the antibody molecule comprises an ensemble antibody homology model derived from a plurality of homology models of the antibody.

In an embodiment, step (d) further comprises removing an antibody molecule-target polypeptide docketing model that exhibits a mode of engagement atypical for a known antibody-antigen complex, e.g., according to a structural filter derived from antibody-antigen crystal structure.

In an embodiment, step (d) comprises generating a plurality of antibody molecule-target polypeptide models.

In an embodiment, step (e) comprises identifying a plurality of sites on the antibody molecule that is capable of being bound by the target polypeptide.

In an embodiment, the site comprises or consists of one or more non-consecutive regions on the antibody molecule. In an embodiment, the site comprises or consists of a consecutive region on the antibody molecule.

In still another aspect, the disclosure features a method of identifying a paratope on an antibody, the method comprising:

(a) generating an antibody-target polypeptide docking model, wherein the antibody-target polypeptide docking model is constrained according to a plurality of enrichment scores determined (e.g., calculated) by a method comprising:
(i) binding the antibody to a plurality of variants of the target polypeptide,
(ii) obtaining (e.g., enriching) variants exhibiting reduced binding to the antibody molecule, and
(iii) determining (e.g., calculating) an enrichment score for each of the plurality of the obtained (e.g., enriched) variants; and

(b) identifying one or more sites on the antibody molecule that is capable of being bound by the target polypeptide based on the antibody-target polypeptide docking model;

thereby identifying a paratope on a target polypeptide.

In an embodiment, the altered binding comprises altered binding affinity, e.g., reduced binding affinity.

In an embodiment, step (a)(i) comprises binding the antibody molecule to a library displaying a plurality of variants of the target polypeptide. In an embodiment, step (a)(i) comprises binding the antibody molecule to a library comprising a plurality of cells expressing (e.g., displaying) a plurality of variants of the target polypeptide. In an embodiment, each of the plurality of cells expresses about one distinct variant of the target polypeptide. In an embodiment, the cell is a eukaryotic cell, e.g., a yeast cell.

In an embodiment, the plurality of variants comprise mutations on one or more surface residues of the target polypeptide. In an embodiment, the plurality of variants comprise distinct mutations of a selected surface residue of the target polypeptide. In an embodiment, the plurality of variants comprise distinct mutations of each of a plurality of selected surface residues of the target polypeptide.

In an embodiment, the plurality of variants comprise single amino acid substitutions, relative to a wild-type amino acid sequence of the target polypeptide. In an embodiment, each of the plurality of variants comprises a single amino acid substitution relative to a wild-type amino acid sequence of the target polypeptide. In an embodiment, the single amino acid substitution occurs at a surface residue of the target polypeptide.

In an embodiment, the altered (e.g., reduced) binding comprises an alteration (e.g., a reduction) of binding detected for the variant and the antibody molecule, relative to the binding detected for a wild-type target polypeptide and the antibody.

In an embodiment, step (a)(ii) comprises obtaining (e.g., enriching) variants exhibiting less than about 80% (e.g., less than about 0.01%, 0.1%, 1%, 2%, 3%, 4%, 5%, 6%, 7%, 8%, 9%, 10%, 20%, 30%, 40%, 50%, 60%, 70%, or 80%) of the binding to the antibody molecule exhibited by a wild-type target polypeptide. In an embodiment, the reduced binding is at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by the wild-type target polypeptide.

In an embodiment, step (a)(ii) comprises obtaining (e.g., enriching) cells exhibiting less than about 80% (e.g., less than about 0.01%, 0.1%, 1%, 2%, 3%, 4%, 5%, 6%, 7%, 8%, 9%, 10%, 20%, 30%, 40%, 50%, 60%, 70%, or 80%) of the binding to the antibody molecule exhibited by a cell comprising a wild-type target polypeptide. In an embodiment, the reduced binding is at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by a cell comprising the wild-type target polypeptide.

In an embodiment, step (a)(ii) comprises performing one or more, e.g., two, three, four, five, six, seven, eight, nine, ten, or more, enrichments for variants exhibiting reduced binding to the antibody molecule.

In an embodiment, the method further comprises, e.g., prior to step (a)(iii), identifying the variants exhibiting altered (e.g., reduced) binding to the antibody molecule, e.g., by sequencing the genes encoding the variants, e.g., by next-generation sequencing.

In an embodiment, step (a)(iii) comprises determining the frequency of occurrence for each of the plurality of the obtained (e.g., enriched) variants. In an embodiment, step (a)(iii) further comprises aggregating the frequency of occurrence of each variant comprising a distinct mutation at a particular residue and/or weighting (e.g., heavily weighting) variants with higher frequencies of occurrence.

In an embodiment, the enrichment score is specific to a single residue of the amino acid sequence of the target polypeptide. In an embodiment, each enrichment score is specific to a different single residue of the amino acid sequence of the target polypeptide.

In an embodiment, the method further comprises repeating steps (a)(i)-(a)(iii) at least once (e.g., once, twice, three times, four times, five times, six times, seven times, eight times, nine times, ten times, or more) with replicates of the plurality of the variants of the target polypeptide, and wherein step (a)(iii) further comprises omitting one or more promiscuous mutations, e.g., mutations for which more than 50% of replicates had an enrichment score of greater than 30% and for which more than 75% of replicates had an enrichment score greater than 15%.

In an embodiment, the antibody molecule-target polypeptide docking model is constrained by adding one or more attractive constraints, optionally, wherein the attractive constraint is for a residue having an enrichment score greater than a first preselected value. In an embodiment, the first preselected value is between 20% and 40%, e.g., between 25% and 35%, e.g., about 25%, about 30%, or about 35%. In an embodiment, the attractive constraint comprises a linearly scaled bonus based on the enrichment score.

In an embodiment, the antibody molecule-target polypeptide docking model is constrained by adding a repulsive constraint for a residue having an enrichment score less than a second preselected value. In an embodiment, the second preselected value is between 5% and 20%, e.g., between 10% and 15%, e.g., about 10%, about 12.5%, or about 15%.

In an embodiment, step (a) comprises generating a docked pose between a model of the antibody molecule and a model of the target polypeptide. In an embodiment, step (a) comprises generating a plurality of docked poses between a model of the antibody molecule and a model of the target polypeptide.

In an embodiment, step (a) further comprises scoring the plurality of docked poses according to a docking algorithm, e.g., SnugDock. In an embodiment, step (a) further comprises selecting a subset of the plurality of docked poses having the highest scores, e.g., the highest scoring 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000 or more docked poses. In an embodiment, step (a) further comprises generating an ensemble docked pose using the selected subset of the plurality of docked poses, and setting the model of the antibody molecule and the model of the target polypeptide in accordance with the ensemble docked pose.

In an embodiment, the model of the antibody molecule comprises an ensemble antibody homology model derived from a plurality of homology models of the antibody.

In an embodiment, step (a) further comprises removing an antibody molecule-target polypeptide docketing model that exhibits a mode of engagement atypical for a known antibody-antigen complex, e.g., according to a structural filter derived from antibody-antigen crystal structure.

In an embodiment, step (a) comprises generating a plurality of antibody molecule-target polypeptide models.

In an embodiment, step (b) comprises identifying a plurality of sites on the target polypeptide that is capable of being bound by the antibody molecule.

In an embodiment, the site comprises or consists of one or more non-consecutive regions on the target polypeptide. In an embodiment, the site comprises or consists of a consecutive region on the target polypeptide.

In an aspect, the disclosure features an antibody molecule for which the epitope on a target polypeptide or the paratope on the antibody molecule for the target polypeptide is identified according to a method described herein.

In an aspect, the disclosure features a nucleic acid molecule encoding an antibody molecule described herein or one or more chains (e.g., VH and/or VL) of an antibody molecule described herein. In another aspect, the disclosure features a vector comprising a nucleic acid molecule described herein. In yet another aspect, the disclosure features a host cell comprising a nucleic acid molecule described herein or a vector described herein. In an aspect, the disclosure features a method of making an antibody molecule, comprising culturing a host cell described herein under conditions suitable for expression of an antibody molecule.

### BRIEF DESCRIPTION OF THE DRAWINGS

FIGS. 1A-1B are a series of diagrams showing positions interrogated on surface of APRIL. (A) Alignment of mouse and human APRIL, with positions interrogated in the deep mutational scanning library highlighted in gray. The chimeric form of APRIL was generated by mutating the 5 positions underlined in red in muAPRIL to the corresponding amino acid found in huAPRIL. (B) Structure of APRIL homotrimer with positions chosen for diversification in the library shaded gray, selected for even coverage of the antigen surface. Nine N-terminal amino acids of APRIL present in the library design but not observed in the APRIL crystal structure are represented (box below structure); two Lys residues were selected for diversification.

FIG. 2 is a graph showing antibody and TACI affinity to APRIL expressed on the surface of yeast. A set of purified anti-APRIL antibodies (2419, 4035, 4540, and 3530), isotype control and TACI were assessed for approximate affinity to APRIL expressed on the surface of yeast. Binding isotherms were used to estimate concentration yielding 80% maximal binding for each antibody, which was used for library enrichment.

FIG. 3 is a series of diagrams showing an overview of epitope mapping with computational docking workflow. A site-saturation library of the APRIL antigen library was generated and expressed by yeast surface display. Antibodies were applied to the yeast library, and FACS enrichment was performed to enrich non-binding members of the library. The enriched library was subjected to NGS to ascertain and count the underlying mutations. Mutation enrichment scores were mapped onto the surface of APRIL to determine putative epitope regions of mapped antibodies. These data were used to constrain antibody-antigen docking, resulting in a cluster of models that are consistent with the mutational profile data. The resultant high-confidence models provide molecular definition of epitope and paratope residues.

FIGS. 4A-4B are a series of graphs showing FACS enrichment of library against multiple antibodies and TACI Flow cytometry analyses of either WT APRIL or library yeast populations are shown before or after enrichment. X-axis represents APRIL surface expression (c-myc) and Y-axis represents antibody/TACI binding. The first column exhibits each antibody or TACI binding to WT APRIL expressed on surfaces of yeast. The second column represents the same binding conditions but against the starting, non-enriched APRIL library. The last column represents the enriched non-binding population after two rounds of FACS enrichment.

FIGS. 5A-5D are a series of diagrams showing mutational profile heatmaps for all tested anti-APRIL antibodies. Enrichment heatmaps (left) were calculated for antibodies (A) 2419, (B) 4035, (C) 4540, and (D) 3530, with residue enrichment scores mapped to the surface of APRIL for each antibody (right).

FIGS. 6A-6C are a series of diagrams showing that epitope mapping of TACI exhibits strong agreement with co-crystal structure. (A) Calculated enrichment heatmap for TACI (left) with values mapped to the surface of APRIL (right). (B) Total enrichment scores for TACI calculated for each position mutated. Epitope residues are defined as those residues that have a heavy atom distance <5 Å from TAC. (C) Structure of TACI in complex with APRIL. Mutated positions on APRIL that make contact with TACI (<5 Å) are shown in spheres shaded according to their total enrichment score.

FIGS. 7A-7B are a series of diagrams showing an example of promiscuous mutations. (A) Enrichment heatmap for residue V132 of APRIL against the panel of tested ligands. Promiscuous mutations to Asp and Glu are highlighted (column), and antibody-specific mutations for 2419 (row) are highlighted. (B) Structure of TACI (dark gray) bound to APRIL (light gray). Residues V132 and E182 of APRIL on different monomers are proximal in the context of the APRIL homotrimer.

FIGS. 8A-8C are a series of diagrams showing the symmetry of the homo-oligomeric assembly of APRIL places equivalent residue positions from different chains in proximity near the apex of the molecule, but not near the equatorial region. Structure of APRIL, colored by chain (A), and by residue position (B and C). Light gray colored residues, at the apex in (B), originate from three different chains of the homotrimer. (C) APRIL homotrimer rotated 90° relative to (B) to show that the equivalent residue positions from different chains are not proximal at the equatorial region.

FIGS. 9A-9D are a series of graphs showing that 3530 binding is uniquely lost to N-terminally truncated APRIL. Antibody 3530 and TACI binding to two different forms of yeast surface-expressed APRIL. Binding to full-length APRIL (residues 96-241) is shown for 3530 (A) and TACI (C). Binding to N-terminally truncated APRIL (residues 106-241) is shown for 3530 (B) and TACI (D).

FIG. 10 is a schematic showing an exemplary computational docking workflow for generating molecularly defined epitope and paratope maps using antibody-antigen docking informed by mutational data derived from deep mutational scanning.

FIGS. 11A-11C are a series of diagrams showing that computational docking of modeled 2419 showed good agreement with the co-crystal structure. (A) Computed Rosetta interface score (Isc) for top 500 docked models of 2419-APRIL complexes vs. interface RMSD relative to the native structure. The top 100 scoring docked models are shaded: light gray (FW RMSD <5 Å), medium gray (5 Å<FW RMSD <10 Å), and dark gray (FW RMSD >10 Å). (B) Overlay of top ranked docked model of 2419-APRIL and native structure of 2419-APRIL, showing high degree of overlap. The docked model and native structure were superimposed based only on the Ca coordinates of the APRIL ligand. (C) Residue enrichment scores experimentally determined for 2419 binding to APRIL. Bars are shaded based on the docking confidence score (frequency that the corresponding residues were found to be contacting 2419 (<5 Å) in the top 100 docked poses). Asterisks indicate contacting positions identified from the native structure.

FIGS. 12A-12B are a series of diagrams showing paratope docking scores and positions mapped to the surface of 2419. (A) Docking confidence scores (paratope) mapped to the surface of 2419. (B) Paratope positions colored in black, derived from the native structure of huAPRIL-2419. Contacts between residues are defined as heavy atom distances <5 Å.

FIGS. 13A-13D are a series of diagrams showing that experimentally-derived constraints incorporated into the computational workflow enabled convergence to near-native modes of engagement. Top row in panel shows APRIL contact residues with 2419, shaded by frequency that residue is in contact with antibody in docked models (heavy atom distance <5 Å). Bottom row shows either top 10 scoring docked 2419-APRIL models or native-structure. (A) Global docking with no experimental constraints. (B) Global docking with incorporation of enrichment-score constraints. (C) Full epitope mapping workflow (constrained global docking, followed by constrained SnugDock, and subsequently using antibody-specific structural filters). (D) Native-structure of 2419-APRIL.

FIGS. 14A-14B are a series of graphs showing the impact of constraints on docking results. Plots of docking interface score computed by Rosetta versus antibody ligand (framework) RMSD (superimposing only on the antigen) compared to native structure of 2419-APRIL complex without using enrichment scores as constraints (A), and using enrichment scores as constraints (B). The top 100 scoring docked models are colored: light gray (FW RMSD <5 Å), medium gray (5 Å<FW RMSD <10 Å), and dark gray (FW RMSD >10 Å), with models not ranked in the top 100 colored gray.

FIGS. 15A-15C is a series of diagrams showing the predicted mode of engagement for each antibody to APRIL. Top panels: APRIL residues are shaded based on the docking confidence score, calculated as the percentage of models where an antigen residue makes contact (heavy atom distance <5 Å) with the antibody. Maps are shown for 2419 (column A), 4035 (column B), and 4540 (column C). Bottom panel: For clarity, a single top scoring antibody pose is shown interacting with ARIL (gray), and occluding binding of TACI (medium gray). Areas of predicted steric clashes on TACI due to antibody binding are indicated in light gray.

FIGS. 16A-16C are a series of diagrams showing that computational models enable rational antibody engineering of species binding specificity. (A) Differences between mouse and human APRIL highlighted on the structure of APRIL. Non-homologous mutations are colored medium gray, and homologous mutations are indicated in dark gray. The docked epitope for each antibody (top ranked model) is shown outlined in light gray. (B) Positions E181 and 1219 are predicted to be proximal to R54 in the heavy chain of APRIL based on docking results. Mutations to arginine and lysine at positions 181 and 219 in the structure of muAPRIL, are predicted to lead to destabilizing interactions with R54 on HCDR2 of 2419. (C) Binding of 2419 and designed variant antibodies to muAPRIL, determined by ELISA. Designed variants contain substitutions: R54D (Design1); T28A_R54D (Design2); L53V_R54D_S56A (Design3).

FIG. 17 is a graph showing binding of 2419 redesigns to human APRIL. ELISA binding results of 2419 and designed variants to human APRIL. Designed variants contained substitutions: R54D (Design1); T28A_R54D (Design2); L53V_R54D_S56A (Design3). Half-maximal binding concentrations were 20 nM (2419), 73 nM (Design1), 63 nM (Design2) and 306 nM (Design3).

### DETAILED DESCRIPTION

### Definitions

As used herein, the term “antibody molecule” refers to a polypeptide that comprises sufficient sequence from an immunoglobulin heavy chain variable region and/or sufficient sequence from an immunoglobulin light chain variable region, to provide antigen specific binding. It comprises full length antibodies as well as fragments thereof, e.g., Fab fragments, that support antigen binding. Typically an antibody molecule will comprise heavy chain CDR1, CDR2, and CDR3 and light chain CDR1, CDR2, and CDR3 sequence. Antibody molecules include human, humanized, CDR-grafted antibodies and antigen binding fragments thereof. In an embodiment an antibody molecule comprises a protein that comprises at least one immunoglobulin variable region segment, e.g., an amino acid sequence that provides an immunoglobulin variable domain or immunoglobulin variable domain sequence.

The VH or VL chain of the antibody molecule can further include all or part of a heavy or light chain constant region, to thereby form a heavy or light immunoglobulin chain, respectively. In one embodiment, the antibody molecule is a tetramer of two heavy immunoglobulin chains and two light immunoglobulin chains.

An antibody molecule can comprise one or both of a heavy (or light) chain immunoglobulin variable region segment. As used herein, the term “heavy (or light) chain immunoglobulin variable region segment,” refers to an entire heavy (or light) chain immunoglobulin variable region, or a fragment thereof, that is capable of binding antigen. The ability of a heavy or light chain segment to bind antigen is measured with the segment paired with a light or heavy chain, respectively. In some embodiment, a heavy or light chain segment that is less than a full length variable region will, when paired with the appropriate chain, bind with an affinity that is at least 20, 30, 40, 50, 60, 70, 80, 90, or 95% of what is seen when the full length chain is paired with a light chain or heavy chain, respectively.

An immunoglobulin variable region segment may differ from a reference or consensus sequence. As used herein, to “differ,” means that a residue in the reference sequence or consensus sequence is replaced with either a different residue or an absent or inserted residue.

An antibody molecule can comprise a heavy (H) chain variable region (abbreviated herein as VH), and a light (L) chain variable region (abbreviated herein as VL). In another example, an antibody comprises two heavy (H) chain variable regions and two light (L) chain variable regions or antibody binding fragments thereof. The light chains of the immunoglobulin may be of types kappa or lambda. In one embodiment, the antibody molecule is glycosylated. An antibody molecule can be functional for antibody dependent cytotoxicity and/or complement-mediated cytotoxicity, or may be non-functional for one or both of these activities. An antibody molecule can be an intact antibody or an antigen-binding fragment thereof.

Antibody molecules include “antigen-binding fragments” of a full length antibody, e.g., one or more fragments of a full-length antibody that retain the ability to specifically bind to an HA target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′) or F(ab′)₂ fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See, e.g., Bird et al. (1988) Science 242:423-426; and Huston et al. (1988) Proc. Natl. Acad. Sci. USA 85:5879-5883. Antibody molecules include diabodies.

As used herein, an “antibody” refers to a polypeptide, e.g., a tetrameric or single chain polypeptide, comprising the structural and functional characteristics, particularly the antigen binding characteristics, of an immunoglobulin. Typically, a human antibody comprises two identical light chains and two identical heavy chains. Each chain comprises a variable region.

The variable heavy (VH) and variable light (VL) regions can be further subdivided into regions of hypervariability, termed “complementarity determining regions” (“CDR”), interspersed with regions that are more conserved, termed “framework regions” (FR). Human antibodies have three VH CDRs and three VL CDRs, separated by framework regions FR1-FR4. The extent of the FRs and CDRs has been precisely defined (see, Kabat, E. A., et al. (1991) Sequences of Proteins of Immunological Interest, Fifth Edition, U.S. Department of Health and Human Services, NIH Publication No. 91-3242; and Chothia, C. et al. (1987) J. Mol. Biol. 196:901-917). Kabat definitions are used herein. Each VH and VL is typically composed of three CDRs and four FRs, arranged from amino-terminus to carboxyl-terminus in the following order: FR1, CDR1, FR2, CDR2, FR3, CDR3, FR4.

The heavy and light immunoglobulin chains can be connected by disulfide bonds. The heavy chain constant region typically comprises three constant domains, CH1, CH2 and CH3. The light chain constant region typically comprises a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

The term “immunoglobulin” comprises various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgD, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure. Light chains are classified as either kappa or lambda (x, X). Each heavy chain class may be bound with either a kappa or lambda light chain.

Suitable antibodies include, but are not limited to, monoclonal, monospecific, polyclonal, poly-specific, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, and antibody fragments.

In an embodiment, an antibody is a humanized antibody. A humanized antibody refers to an immunoglobulin comprising a human framework region and one or more CDR's from a non-human, e.g., mouse or rat, immunoglobulin. The immunoglobulin providing the CDR's is often referred to as the “donor” and the human immunoglobulin providing the framework often called the “acceptor,” though In an embodiment, no source or no process limitation is implied. Typically a humanized antibody comprises a humanized light chain and a humanized heavy chain immunoglobulin.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay (1988) Ann. Rev. Immunol. 6:381-405).

As used herein, an “immunoglobulin variable domain sequence” refers to an amino acid sequence that can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that comprises an immunoglobulin variable domain sequence can associate with another immunoglobulin variable domain sequence to form a target binding structure (or “antigen binding site”), e.g., a structure that interacts with the target antigen.

As used herein, the term antibodies comprises intact monoclonal antibodies, polyclonal antibodies, single domain antibodies (e.g., shark single domain antibodies (e.g., IgNAR or fragments thereof)), multispecific antibodies (e.g., bi-specific antibodies) formed from at least two intact antibodies, and antibody fragments so long as they exhibit the desired biological activity. Antibodies for use herein may be of any type (e.g., IgA, IgD, IgE, IgG, IgM).

The antibody or antibody molecule can be derived from a mammal, e.g., a rodent, e.g., a mouse or rat, horse, pig, or goat. In an embodiment, an antibody or antibody molecule is produced using a recombinant cell. In an embodiment an antibody or antibody molecule is a chimeric antibody, for example, from mouse, rat, horse, pig, or other species, bearing human constant and/or variable regions domains.

As used herein, the term “variant” refers to a polypeptide comprising an amino acid sequence comprising one or more mutations (e.g., amino acid substitutions, deletions, insertions, or any other mutation known in the art) relative to the amino acid sequence of a wild-type form of a target polypeptide. In some instances, a variant includes about one amino acid substitution, e.g., to a surface residue, relative to the amino acid sequence of the wild-type form of the target polypeptide. By “wild-type,” as used herein, is meant a form of a target polypeptide comprising a reference amino acid sequence. In some instances, a wild-type target polypeptide comprises an amino acid sequence that occurs in nature (e.g., an endogenous sequence from a living organism). In other instances, a wild-type target polypeptide comprises any reference amino acid sequence (e.g., a consensus amino acid sequence, e.g., compiled from a plurality of naturally occurring versions of the target polypeptide).

As used herein, the term “target polypeptide” refers to any polypeptide that is desirably bound by an antibody molecule. A target polypeptide may include one or more epitope regions on its surface that are contacted by the antibody molecule. The methods described herein may be used to identify such epitope regions. A target polypeptide may bind to one or more paratope regions on the antibody molecule, which can likewise be identified according to the methods herein. In some instances, the terms “target polypeptide” and “antigen” may be used interchangeably.

As used herein, the term “epitope” refers to a portion of a target polypeptide (e.g., as described herein) contacted by another polypeptide, e.g., an antibody molecule, e.g., by one or more CDRs of the antibody molecule and/or one or more framework residues of the antibody molecule. In some instances, an epitope comprises one or more surface residues of the target polypeptide. A “surface residue” of a protein or polypeptide is generally an amino acid residue positioned on the exterior surface of the protein or polypeptide, e.g., such that at least a portion of the amino acid (e.g., the side chain) is accessible to another molecule external to the protein or polypeptide. Epitope residues may be contiguous or may not be contiguous. In some instances, an epitope comprises a plurality of regions or patches that contact the antibody molecule. In certain instances, two or more of the regions or patches are not contiguous or in close physical proximity, e.g., a conformational epitope.

As used herein, the term “paratope” refers to a portion of an antibody molecule contacted by a target polypeptide (e.g., as described herein), or a variant thereof. A paratope may comprise one or more CDRs of the antibody molecule and/or one or more framework residues of the antibody molecule. In some instances, a paratope comprises one or more surface residues of the antibody molecule. Paratope residues may be contiguous or may not be contiguous. In some instances, a paratope comprises a plurality of regions or patches that contact the target polypeptide. In certain instances, two or more of the regions or patches are not contiguous or in close physical proximity.

As used herein, the term “model” generally refers to a structure, e.g., a three-dimensional model, e.g., a simulated and/or calculated structure, of one or more molecules (e.g., a target polypeptide and/or an antibody molecule). In some instances, the term “modeling” is used to refer to the process of generating a model. A model can be generated, for example, by X-ray crystallography or by computational methods, e.g., as described herein. A model can be generated by aggregating information from one or more other models. In some instances, a model comprises a plurality of other models. In some instances, a model is generated using a plurality of other models. A “model of” an entity refers to a model representing the structure of the entity. The term “docking model,” as used herein, generally refers to a model (e.g., a three-dimensional model) for the interaction between an antibody molecule and a target polypeptide, or a variant thereof. In some instances, a docking model comprises a model of the antibody molecule and a model of the target polypeptide, or variant thereof. In some instances, a docking model shows the points of contact between the antibody molecule and the target polypeptide, or variant thereof.

The terms “purified” and “isolated” as used herein in the context of an antibody molecule, e.g., a antibody, a immunogen, or generally a polypeptide, obtained from a natural source, refers to a molecule which is substantially free of contaminating materials from the natural source, e.g., cellular materials from the natural source, e.g., cell debris, membranes, organelles, the bulk of the nucleic acids, or proteins, present in cells. Thus, a polypeptide, e.g., an antibody molecule, that is isolated includes preparations of a polypeptide having less than about 30%, 20%, 10%, 5%, 2%, or 1% (by dry weight) of cellular materials and/or contaminating materials. The terms “purified” and “isolated” when used in the context of a chemically synthesized species, e.g., an antibody molecule, or immunogen, refers to the species which is substantially free of chemical precursors or other chemicals which are involved in the syntheses of the molecule.

Calculations of “homology” or “sequence identity” or “identity” between two sequences (the terms are used interchangeably herein) can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

### Cell Display Assays

The methods of the invention generally involve displaying variants of a target polypeptide on cells (e.g., yeast cells) and assessing the binding capacity of an antibody for the variants of the target polypeptide, e.g., by enriching the population of cells displaying variants exhibiting reduced binding (e.g., reduced binding affinity) to the antibody. Examples of cells that can be used according to the methods described herein include, without limitation, eukaryotic cells (e.g., fungal cells, e.g., yeast cells; mammalian cells, e.g., CHO cells or human cells) or prokaryotic cells (e.g., bacterial cells, e.g., E. coli cells). In an embodiment, the cells are yeast cells.

In an embodiment, epitope mapping data are derived from deep mutational scanning of libraries of target polypeptides (also referred to herein as antigens), which addresses the low-throughput nature of typical mutagenesis genotype-phenotype studies and enables the simultaneous testing of many (e.g., hundreds, thousands, or tens of thousands) of mutational variants for impact on function. The throughput of the method can enable a more comprehensive sampling of surface residues as well as multiple distinct mutations per residue (i.e., not only mutations to alanine), and therefore a more sensitive and complete mapping of epitopes, including conformational epitopes.

In an embodiment, variants of a target polypeptide are expressed on the surface of cells (e.g., yeast cells), e.g., by fusion through a linker sequence to an endogenous cell surface protein, e.g., the yeast protein Aga2. In an embodiment, e.g., in which the target polypeptide normally forms multimers, a long flexible linker sequence (e.g., a linker comprising at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids) between the linker and a given variant may provide sufficient proximity for neighboring target polypeptide molecules to associate, thereby presenting native quaternary structure. In an embodiment, the linker comprises 35 amino acids.

In an embodiment, the method comprises one or more steps described in the Examples. In an embodiment, the method is performed in accordance with the Examples.

### Target Polypeptide Variants

In an embodiment, a population of variants of a target polypeptide are tested for binding capacity and/or binding affinity to an antibody of interest. A population of target polypeptide variants may, In an embodiment, include mutations to surface residues of the target polypeptide, which can be used to identify surface regions of the polypeptide that contact the antibody of interest, e.g., using epitope mapping methods described herein or as known in the art. For example, each of the population of variants may include at least one (e.g., 1, 2, 3, 4, 5, 6, 7, 8, 9, 10, 11, 12, 13, 14, 15, or more) amino acid substitutions at a surface residue. In an embodiment, the population includes variants having a distribution of surface residue mutations suitable for identifying regions of contact between the antibody and the target polypeptide at a desired resolution.

A library of such variants can be generated, for example, by deep mutational scanning, e.g., as described herein. In an embodiment, a library of variants is designed to maximize informational output for epitope mapping derived from deep mutational scanning, e.g., by first identifying all surface residues that are unlikely to have significant detrimental effects on protein structure when mutated. In an embodiment, surface residues may be selected based on relative sidechain surface accessibility (e.g., using Discovery Studio). In an embodiment, residues exhibiting relative sidechain surface accessibility of greater than about 25% (e.g., greater than about 5%, 10%, 15%, 20%, 25%, 30%, 35%, 40%, 45%, 50%, 60%, 70%, 80%, 90%, 95%, or 99%) are selected for mutation. In an embodiment, residues tolerant to mutation may be identified, e.g., by visual inspection and/or their interactions with and/or proximity to neighboring residues. In an embodiment, all surface residues of a target polypeptide are identified as a set of residues with potential to make direct contact with bound antibodies. In an embodiment, Pro and/or Gly residues are excluded from consideration, as mutating such residues may be more likely to perturb the protein structure, which may lead to false positives for epitope mapping through an indirect effect on binding.

In an embodiment, a set of residues to be mutated is selected for even coverage across the surface of the target polypeptide. Residues can, in an embodiment, be visually curated to ensure even coverage, for selection of a set of surface positions for mutation spanning the entire surface. In an embodiment, additional N-terminal and/or C-terminal residues may be selected for mutation. In an embodiment, one or more residues not resolved in an X-ray crystallography structure of the target polypeptide may be selected for mutation. In an embodiment, at least about 5, 10, 20, 30, 40, 50, 60, 70, 80, 90, 100, 110, 120, 130, 140, 150, 160, 170, 180, 190, 200, 225, 250, 275, 300, 350, 400, 450, 500, 550, 600, 650, 700, 750, 800, 850, 900, 950, or 1000 residues are selected for mutation.

In an embodiment, a single-site saturation mutagenesis library representing the selected positions is synthesized, e.g., using NNK degeneracy. Deep sequencing of the synthesized library can be used to verify the presence of mutations at intended positions. In an embodiment, linkage of genotype-phenotype is maintained by coupling single mutations to phenotype, e.g., using a non-combinatorial, site-saturation library.

### Library Selections

A library of target polypeptide variants can be transformed into cells and assessed for impact of the mutations on binding. In an embodiment, a library is transformed into yeast cells. Preferably, the transformation provides a thorough (e.g., about 5000-fold, e.g., about 100-fold, 500-fold, 1000-fold, 2000-fold, 3000-fold, 4000-fold, 5000-fold, 6000-fold, 7000-fold, 8000-fold, 9000-fold, 10,000-fold, or more) oversampling of the unique genetic diversity (e.g., 32 possible codons at each position). In an embodiment, sensitivity for detection of mutations which disrupt antibody binding is maximized, e.g., using a concentration of antibody corresponding to about 80% (e.g., about 50%, 60%, 70%, 80%, 90%, or 100%) maximal binding for the wild-type target polypeptide displayed on cells. In an embodiment, antibody binding is used to distinguish variants that exhibit different binding properties. In an embodiment, variants exhibiting reduced binding are selected for. In an embodiment, variants exhibiting increased binding are selected for.

In an embodiment, fluorescence activated cell sorting (FACS) is used to select for (e.g., enrich) variants exhibiting different binding properties (e.g., reduced or increased binding relative to the wild-type target polypeptide). In an embodiment, variants exhibiting reduced binding relative to the wild-type target polypeptide, e.g., reduced binding of at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by a cell comprising the wild-type target polypeptide, are selected. In an embodiment, variants exhibiting increased binding relative to the wild-type target polypeptide, e.g., increased binding of at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by a cell comprising the wild-type target polypeptide, are selected. In an embodiment, at least two (e.g., at least 2, 3, 4, 5, 6, 7, 8, 9, 10, 11, 12, 13, 14, 15, 16, 17, 18, 19, 20, or more) rounds of FACS enrichment (e.g., enrichment of an expressing but non-binding population) is performed. In an embodiment, at least about 1000 cells (e.g., at least about 1000, 2000, 3000, 4000, 5000, 6000, 7000, 8000, 9000, 10,000, 15,000, 20,000, 25,000, 30,000, 35,000, 40,000, 45,000, 50,000, 60,000, 70,000, 80,000, 90,000, or 100,000 cells) are collected for a given sample. In an embodiment, at least about 30,000 cells are collected for a given sample. In certain embodiment, the FACS enrichment yields populations lacking any significant binding ability to their respective antibodies.

In an embodiment, cells (e.g., yeast cells) expressing a library of target polypeptide variants are exposed to an antibody, e.g., at a concentration corresponding to about 80% (e.g., about 50%, 60%, 70%, 80%, 90%, or 100%) maximal binding for the antibody to the target polypeptide, e.g., based on antibody titration binding experiments with cells (e.g., yeast cell) expressing the wild-type target polypeptide.

### Deep Sequencing and Bioinformatics

In an embodiment, selected variants from binding experiments are subjected to deep sequencing, e.g., to ascertain and quantify the underlying genotypes. In an embodiment, sequencing reads having a quality score below a predetermined threshold (e.g., a quality score of less than about 30) are removed from the data set. In an embodiment, reads comprising an insertion and/or a deletion mutation are removed from the data set. In an embodiment, reads comprising a number of base substitutions above a predetermined threshold (e.g., greater than about 5, 6, 7, 8, 9, 10, 11, 12 13, 14, 15, 20, 30, 40, or 50 base substitutions) are removed from the data set. In an embodiment, reads comprising internal stop codons, mutations at unintended positions, and/or more than one amino acid substitution relative to the wild-type target polypeptide are removed from the data set. In an embodiment, nucleotide reads are converted to amino acid reads. In an embodiment, mutant variants in which fewer than a predetermined threshold number of reads (e.g., fewer than about 10, 20, 30, 40, 50, 60, 70, 80, 90, 100, 110, 120, 130, 140, 150, 160, 170, 180, 190, 200, 250, 300, 400, 500, 600, 700, 800, 900, or 1000 reads) are removed from the data set.

In an embodiment, a bioinformatic analysis is performed to calculate the levels of enrichment sequenced variants against the antibody. In an embodiment, variants enriched in the non-binding population relative to the starting library represent mutations that reduce antibody binding affinity. In an embodiment, variants enriched in an elevated binding population relative to the starting library represent mutations that increase antibody binding affinity. Mechanisms contemplated to cause reduced binding include, for example, direct effects, such as change in residue side chains making direct contact with the antibody, and indirect effects, such as by change in local or global protein structure unrelated to a contact residue. Structurally disruptive mutations may impact binding of antibodies with divergent epitopes. In an embodiment, a panel of antibodies is incorporated with different binding modes (e.g., determined using competition binding experiments) to aid computational efforts to discern mutations likely causing indirect effects on antibody binding.

### Enrichment Scores

An enrichment score, representing the level of enrichment of a particular variant after library selection, may be calculated for each variant, e.g., based on selection data generated as described herein. In an embodiment, an enrichment score for each mutation is calculated as follows: for each sample collected in a non-binding pool, the position-dependent frequency of occurrence of a mutation in a sample is normalized by the frequency of occurrence of that mutation in the expresser pool, and scaled by the fraction of variants found in the non-binding pool as follows:

$${E}_{p , aa}^{s} = N {B}^{s} ( \frac{{f}_{p , aa}^{s}}{{f}_{p , aa}^{wt}} )$$

wherein E_{p,aa}^{s} is the enrichment score for a given amino acid (aa) at positon (p) for sample (s), NB^{s} is the fraction (pool size) of variants found in the non-binding pool, and f_{p,aa} is the observed positional frequency of the amino acid in either a sample (s) or the expresser pool (wt). In an embodiment, the enrichment score represents the fraction of a mutation from the expresser pool that is found in the non-binding pool (e.g., represented here as a percentage).

In an embodiment, the fraction of each mutation in the non-binding pool is calculated based on the sequencing results. In an embodiment, for each mutation, the frequency of occurrence found in the non-binding pool relative to the frequency found in the expresser pool is used to calculate an enrichment score. In an embodiment, the enrichment score calculated for a variant represent the fraction of a particular mutation that was found in the non-binding pool, e.g., with a range of 0-100%. In an embodiment, mutations to Pro, Gly, or Cys were omitted from consideration due to their higher propensity to alter tertiary or quaternary structure. In an embodiment, site-specific mutations predicted to insert or remove a glycosylation site were omitted from consideration. In an embodiment, a residue enrichment score is calculated by aggregating the enrichment scores for each mutation for a particular residue, e.g., in a manner that more heavily weights mutations with high enrichment scores. Residues with higher enrichment scores generally reflect greater sensitivity to mutation with respect to binding, e.g., indicating that this position is more likely to be part of the epitope. In an embodiment, enrichment scores are then mapped to the surface of the target polypeptide, and positions with high enrichment scores (e.g., on surface patches of the target polypeptide) are designated as part of the epitope.

Without wishing to be bound by theory, certain mutations may show above-background enrichment scores across a plurality of systems, often with a low to mid enrichment score value. This promiscuous effect on binding for many antibodies may, in some instances represent false positives, e.g., caused by reduction in binding through indirect mechanisms. Thresholds for identifying promiscuous mutations for removal from epitope mapping can be empirically determined, e.g., based on inspection of enrichment maps for all samples. In an embodiment, mutations in which more than about 50% (e.g., about 30%, 40%, 45%, 50%, 55%, 60%, or 70%) of samples had an enrichment score greater than about 30% (e.g., about 20%, 25%, 30%, 35%, 40%, 45%, or 50%) and, optionally, in which more than about 75% (e.g., about 50%, 60%, 70%, 75%, 80%, 90%, or 95%) of samples had an enrichment score greater than 15% (e.g., about 5%, 10%, 15%, 20%, 25%, or 30%), are considered false positives and are removed for epitope determination. In an embodiment, promiscuous mutations can be identified by structural analysis of the antibody-antigen complex, e.g., to show that such residues are not involved in antibody-antigen contact or that a mutation may destabilize, e.g., secondary, tertiary, or quaternary structures (e.g., by electrostatic attraction or repulsion).

In an embodiment, enrichment scores and epitope maps can be calculated for a plurality of biological replicates (e.g., at least 2, 3, 4, 5, 6, 7, 8, 9, 10, 15, 20, 25, or 30 biological replicates), e.g., to assess reproducibility. In an embodiment, the accuracy of enrichment score results can be validated, e.g., by comparing them to a co-crystal structure for the target polypeptide with the antibody or a comparable surrogate thereof (e.g., a ligand or receptor for the target polypeptide).

In an embodiment, an aggregate of mutational data for a given amino acid position on the target polypeptide can be generated, e.g., for assessment that the amino acid position is part of the epitope. In an embodiment, a total enrichment score is calculated for each residue, e.g., by aggregating the effect of each mutation at the corresponding position. In an embodiment, enrichment scores are calculated as follows:

$${E}_{p}^{s} = \sqrt{\frac{{\sum }_{i}^{{N}_{p , aa}} {( {E}_{p , aa}^{s} )}^{2}}{{N}_{p , aa}}}$$

wherein N_{p,aa} is the number of amino acid mutations at a given position after filtering. Generally, a calculated total residue enrichment score more heavily weights the effect of mutations that show a large enrichment score and/or down-weights contributions from mutations that show low enrichment scores. This may ensure that positions that show low levels of enrichment for multiple mutations, which may be due to noise, do not mask the signal from positions which may have a smaller number of mutations but with higher enrichment. In an embodiment, once total enrichment scores are calculated for each position, the total enrichment scores can be mapped onto protein surfaces to facilitate visualization of enrichment epitope maps.

### Computational Modeling of Antibody-Antigen Complexes

The methods described herein generally involve identifying one or more epitope regions or sites on a target polypeptide that are bound by an antibody of interest, or an antigen-binding fragment thereof. Such epitope regions may be identified, for example, using computational modeling of an antibody-antigen complex (e.g., using a docking algorithm), which can be informed, e.g., by the results of a cell display assay, e.g., as described herein. In an embodiment, the results of a cell display assay (e.g., enrichment scores, e.g., as described herein) are incorporated as a constraint into a docking algorithm. In an embodiment, the method comprises one or more steps described in the Examples. In an embodiment, the method is performed in accordance with the Examples.

### Antibody-Antigen Docking

Generally, a multi-step docking approach can be implemented to generate an antibody-antigen model that preferably (1) incorporates experimentally derived epitope mapping as a constraint, (2) uses an ensemble of antibody models to better account for uncertainty in homology modeling, and (3) utilizes the large amount of antibody-specific structural knowledge to more effectively identify docked models that exhibit features characteristic of antibody-antigen complexes. In an embodiment, residue enrichment scores, e.g., obtained from deep mutational scanning data as described herein, are used as constraints for an antibody-antigen global docking algorithm, e.g., which samples antibody engagement over the entirety of the antigen surface. In an embodiment, the constraints are used to designate antibody-antigen poses as favored when making maximal contact with high enrichment positions, and/or to designate antibody-antigen poses as disfavored when contacting positions that were determined to be tolerant to mutation.

In an embodiment, antibody homology models (e.g., for using in generating antibody-antigen docking models) are generated, e.g., using algorithms and/or protocols known in the art (e.g., Rosetta antibody homology modeling, e.g., Rosette 3.8, or BioLuminate Schrödinger). In an embodiment, the antibody homology models are varied, e.g., in the conformation of a CDR region (e.g., an HCDR1, HCDR2, HCDR3, LCDR1, LCDR2, and/or LCDR3). In an embodiment, the models vary primarily in the conformation of HCDR3 (e.g., in the HCDR3 loop).

Docking can be performed, for example, using an ensemble of different antibody homology models as input. In an embodiment, the docking program PIPER is used for global docking, e.g., using a customized score function derived from known antibody-antigen complexes. In an embodiment, constraints from enrichment scores are used during generation of docked models, e.g., utilizing attractive and/or repulsive constraints to alter the docking results. This permits epitope mapping approaches that identify residues with high enrichment scores (e.g., transformed into attractive constraints for docking), and/or identify residues with low enrichment scores, which would not be expected to be part of the epitope (e.g., transformed into repulsive constraints). In an embodiment, constraints are generated only using residues with either high or low enrichment scores, e.g., such that residues with intermediate enrichment scores are not constrained during docking. In an embodiment, data generated from a panel of antibodies are used to identify mutations that impact binding of many antibodies and are thus more likely to be false positives. Such false positives can, in an embodiment, be excluded from consideration when generating constraints. In an embodiment, a docking approach as described herein does not rely on an absolute cutoff for deciding whether an enriched position should be included as part of an epitope.

In an embodiment, constraints are incorporated into the docking run as follows: attractive constraints are added for sites with residue enrichment scores greater than about 30% (e.g., greater than about 20%, 25%, 30%, 35%, 40%, 45%, or 50%), with attractive bonuses, e.g., linearly scaled from, e.g., 0.35 to 0.99, based on the enrichment score. In an embodiment, repulsive constraints are added for sites with residue enrichment scores less than about 12.5% (e.g., about 5%, 10%, 11%, 12%, 12.5%, 13%, 14%, 15%, 20%, 25%, or 30%). In an embodiment, global docking is performed for each of a series of input antibody homology models (e.g., a series of at least about 5, 10, 15, 20, 25, 30, 40, 50, or more input antibody homology models). In an embodiment, a total of at least about 50, 100, 200, 300, 400, 500, 600, 700, 800, 900, or 1000 docked poses are generated. In an embodiment, about 30 poses (e.g., about 10, 15, 20, 25, 30, 35, 40, 45, or 50 poses) representing cluster centers are obtained for each sample.

In an embodiment, an epitope map score is calculated to assess the level of agreement between each docked model and the experimentally determined enrichment scores. In an embodiment, the epitope map score is calculated using the following equation:

$$E S = {\sum }_{p = 1}^{N} {c}_{p}$$

$${c}_{p} = \{ \begin{matrix}{E}_{p} - 30 , if {E}_{p} > 30 \\ 12.5 - {E}_{p} , if {E}_{p} < 1 \; 2.5\end{matrix}$$

wherein ES is the epitope map score, N is the number of mutated sites, cₚ is the constraint at position p, and Eₚ is the enrichment score at position p. In an embodiment, docked models are ranked by the epitope map score. In an embodiment, a certain number of the top models are selected (e.g., the top 5, 10, 15, 20, 25, 30, 35, 40, 45, 50, 60, 70, 80, 90, 100 or more models).

In an embodiment, the antibody-antigen docking involves generating an ensemble docking model in which a plurality of antibody homology models are docked to one or more models of the antigen. In an embodiment, the plurality of antibody homology models are docked to one model of the antigen. In an embodiment, the plurality of antibody homology models are docked to a plurality of models of the antigen. In an embodiment, an ensemble of top solutions is used to represent the antibody-antigen complex. In another embodiment, the single top ranked model from the docking workflow is selected to represent the docked complex.

In an embodiment, docked poses generated as described herein can be refined, e.g., using a local docking algorithm (e.g., SnugDock). In an embodiment, the local docking algorithm refines the docked poses, e.g., by exploring small rigid body movements, allowing repacking of sidechains, remodeling of CDR regions (e.g., HCDR1, HCDR2, HCDR3, LCDR1, LCDR2, and/or LCDR3; preferably HCDR2 and/or HCDR3), refinement of CDR loops (e.g., HCDR1, HCDR2, HCDR3, LCDR1, LCDR2, and/or LCDR3; preferably HCDR2 and/or HCDR3), and/or resampling of VH/VL orientation. In an embodiment, constraints from enrichment scores are used in local docking (e.g., as described above for global docking), e.g., utilizing attractive and/or repulsive constraints to alter local docking results. In an embodiment, residues with high enrichment scores are transformed into attractive constraints for docking. In an embodiment, residues with low enrichment scores are transformed into repulsive constraints.

In an embodiment, a set of antibody-specific structural filters, e.g., derived from a set of available antibody-antigen crystal structures, are applied to remove models exhibiting modes of engagement atypical for known antibody-antigen complexes. In an embodiment, the structural filters are selected from those listed in Table 1 (e.g., at least 1, 2, 3, 4, 5, 6, 7, 8, 9, 10, 11, 12, 13, 14, or all of the structural filters listed in Table 1). In an embodiment, residues are considered contacting if a pair of heavy atoms in both residues is <5 Å apart.

TABLE 1
Exemplary antibody-antigen structural filters used to filter docked poses

| Filter | Description |
|---|---|
| SASA < 1250 | Interface SASA calculated using Rosetta |
| nEpitope <= 12 | Number of antigen residues contacting antibody |
| nEpitopeCDR <= 9 | Number of antigen residues being contacted by a CDR residue |
| nParatope <= 16 | Number of antibody residues contacting the antigen |
| nParatopeCDRs <= 12 | Number of antibody CDR residues contacting the antigen |
| percentCDR <= 0.55 | nParatopeCDRs/nParatope |
| nPairwiseContacts < 40 | Number of pairwise contacts made between antibody and antigen |
| nCDRPairwiseContacts < 25 | Number of pairwise contacts (dist <5A) made between antibody |
| | CDR residues and the antigen |
| nCDRLoops < 3 | Number of CDR Loops with a residue contacting the antigen |
| diffCDR31 < −2 | Number of residues in CDR3 (H + L) − number of residues in CDR1 |
| | (H + L) contacting the antigen |
| nHCDR3 + nLCDR3 < 5 | Number of residues in HCDR3 and LCDR3 contacting antigen |
| ContactDensity < 0.8 | nPairwiseContacts/(nEpitope + nParatope) |
| CDRContactDensity < 0.75 | nCDRPairwiseContacts/(nParatopeCDRs + nEpitopeCDRs) |
| LoopDensity < 2.25 | nParatopeCDRS/nCDRLoops |
| Score_EPII < 0.03 | Score based on antibody-antigen pairwise propensities |

In an embodiment, the structures of at least about 100 (e.g., about 100, 150, 200, 250, 300, 350, 400, 450, 500, or more) available antibody-antigen complexes are used to generate the structural filters. In an embodiment, complexes with missing regions near the interface and/or complexes with ligands or post-translational modifications at the interface are removed. Generally, for the set of antibody-antigen complexes to be used for generating the structural filters, distributions of structural features for key interface properties are calculated (e.g., the number of CDR and/or framework residues engaging the epitope, the number and type of CDR loops involved in interactions, the number of epitope residues, the buried surface area, and/or pairwise residue propensities). In an embodiment, thresholds for one or more of the above interface properties are selected such that a predetermined quantity (e.g., at least about 85%, 90%, 95%, 96%, 97%, 98%, 99%, or 99.9%) of the structures fail no more than one of the structural filters.

In an embodiment, interface properties are calculated for each of the docked models. In an embodiment, models that fail more than one of the structural filters are removed. In an embodiment, the remaining docked models are filtered based on an epitope map score (e.g., as described herein). In an embodiment, docked models are allowed to make contact with a small number of residues with low enrichment scores. In an embodiment, models with enrichment scores less than about 80% of the maximum observed epitope map score are removed. In an embodiment, the remaining docked models are ranked based on their interface energy (Isc), e.g., as calculated using Rosetta.

In an embodiment, specific knowledge of antibody-antigen complexes derived from the large number of structures available is used to identify near-native models. Docking algorithms generally utilize physics-based scoring functions that have been parameterized to be general for protein-protein interactions. In an embodiment, a curated database of antibody-antigen structures is generated and a distribution of structural features is calculated, e.g., including the buried surface area, the number and type of CDR residues engaging the antigen, the fraction of paratope residues coming from CDR loops, and/or pairwise residue propensities. Candidate docked models can then be assessed on theses structural features, while models with atypical interfaces can be removed from consideration.

### Antibody Engineering

In addition to identifying the epitope residues consistent with the crystal structure, the docked models can also provide paratope information. This can be utilized for further engineering of the antibody, for example, in humanization, affinity maturation, alteration of antigen binding specificity, and/or improvement of biophysical properties (e.g., aggregation propensity). In an embodiment, paratopic residues and/or regions can be identified using the antibody-antigen docking models generated as described herein.

In an embodiment, identified paratope residues can be engineered to modulate an activity or alter a structural characteristic of the antibody. For example, paratope residues can be modified to increase or decrease cross-species reactivity for the target polypeptide (e.g., mouse and human, cynomolgus and human, mouse and cynomolgus, or any other pairwise combination of species), and/or to increase or decrease cross-reactivity for the target polypeptide and one or more related proteins.

In an embodiment, the disclosure herein includes an antibody molecule engineered by a method described herein. In an embodiment, the disclosure herein includes a composition (e.g., a pharmaceutical composition) comprising an antibody molecule engineered by a method described herein and a pharmaceutically acceptable carrier. In an embodiment, the disclosure herein includes a nucleic acid molecule encoding an antibody molecule engineered by a method described herein. In an embodiment, the disclosure herein includes a vector comprising a nucleic acid molecule encoding an antibody molecule engineered by a method described herein. In an embodiment, the disclosure herein includes a cell (e.g., a host cell) comprising nucleic acid molecule encoding an antibody molecule engineered by a method described herein. In an embodiment, the disclosure herein includes a method of making an antibody molecule engineered by a method described herein.

The present disclosure also includes any of the following numbered paragraphs:

1. A method of identifying an epitope on a target polypeptide, the method comprising:

(a) binding an antibody molecule to a plurality of variants of the target polypeptide;

(b) obtaining (e.g., enriching) a plurality of variants exhibiting reduced binding (e.g., reduced binding affinity) to the antibody molecule;

(c) determining (e.g., calculating) an enrichment score for each of the plurality of the obtained (e.g., enriched) variants;

(d) generating an antibody molecule-target polypeptide docking model, wherein the antibody molecule-target polypeptide docking model is constrained according to the enrichment scores; and

(e) identifying a site on the target polypeptide that is capable of being bound by the antibody molecule based on the antibody molecule-target polypeptide docking model;

thereby identifying an epitope on a target polypeptide.

2. The method of paragraph 1, wherein step (a) comprises binding the antibody molecule to a library displaying a plurality of variants of the target polypeptide.

3. The method of paragraph 1 or 2, wherein step (a) comprises binding the antibody molecule to a library comprising a plurality of cells expressing (e.g., displaying) a plurality of variants of the target polypeptide.

4. The method of paragraph 3, wherein each of the plurality of cells expresses about one distinct variant of the target polypeptide.

5. The method of paragraph 3 or 4, wherein the cell is a eukaryotic cell, e.g., a yeast cell.

6. The method of any of the preceding paragraph s, wherein the plurality of variants comprise mutations on one or more surface residues of the target polypeptide.

7. The method of any of the preceding paragraph s, wherein the plurality of variants comprise distinct mutations of a selected surface residue of the target polypeptide.

8. The method of any of the preceding paragraph s, wherein the plurality of variants comprise distinct mutations of each of a plurality of selected surface residues of the target polypeptide.

9. The method of any of the preceding paragraph s, wherein the plurality of variants comprise single amino acid substitutions, relative to a wild-type amino acid sequence of the target polypeptide.

10. The method of any of the preceding paragraphs, wherein each of the plurality of variants comprises a single amino acid substitution relative to a wild-type amino acid sequence of the target polypeptide.

11. The method of paragraph 9 or 10, wherein the single amino acid substitution occurs at a surface residue of the target polypeptide.

12. The method of any of the preceding paragraphs, wherein the reduced binding comprises a reduction of binding detected for the variant and the antibody molecule, relative to the binding detected for a wild-type target polypeptide and the antibody.

13. The method of any of the preceding paragraphs, wherein step (b) comprises obtaining (e.g., enriching) variants exhibiting less than about 80% (e.g., less than about 0.01%, 0.1%, 1%, 2%, 3%, 4%, 5%, 6%, 7%, 8%, 9%, 10%, 20%, 30%, 40%, 50%, 60%, 70%, or 80%) of the binding to the antibody molecule exhibited by a wild-type target polypeptide.

14. The method of paragraph 13, wherein the reduced binding is at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by the wild-type target polypeptide.

15. The method of any of the preceding paragraphs, wherein step (b) comprises obtaining (e.g., enriching) cells exhibiting less than about 80% (e.g., less than about 0.01%, 0.1%, 1%, 2%, 3%, 4%, 5%, 6%, 7%, 8%, 9%, 10%, 20%, 30%, 40%, 50%, 60%, 70%, or 80%) of the binding to the antibody molecule exhibited by a cell comprising a wild-type target polypeptide.

16. The method of paragraph 15, wherein the reduced binding is at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by a cell comprising the wild-type target polypeptide.

17. The method of any of the preceding paragraphs, wherein step (b) comprises performing one or more, e.g., two, three, four, five, six, seven, eight, nine, ten, or more, enrichments for variants exhibiting reduced binding to the antibody molecule.

18. The method of any of the preceding paragraphs, further comprising, e.g., prior to step (c), identifying the variants exhibiting reduced binding to the antibody molecule, e.g., by sequencing the genes encoding the variants, e.g., by next-generation sequencing.

19. The method of any of the preceding paragraphs, wherein step (c) comprises determining the frequency of occurrence for each of the plurality of the obtained (e.g., enriched) variants.

20. The method of paragraph 19, wherein step (c) further comprises aggregating the frequency of occurrence of each variant comprising a distinct mutation at a particular residue and/or heavily weighting variants with higher frequencies of occurrence.

21. The method of any of the preceding paragraphs, wherein the enrichment score is specific to a single residue of the amino acid sequence of the target polypeptide.

22. The method of any of the preceding paragraphs, wherein each enrichment score is specific to a different single residue of the amino acid sequence of the target polypeptide.

23. The method of any of the preceding paragraphs, further comprising repeating steps (a)-(c) at least once (e.g., once, twice, three times, four times, five times, or more) with replicates of the plurality of the variants of the target polypeptide, and wherein step (c) further comprises omitting one or more promiscuous mutations, e.g., mutations for which more than 50% of replicates had an enrichment score of greater than 30% and for which more than 75% of replicates had an enrichment score greater than 15%.

24. The method of any of the preceding paragraphs, wherein the antibody molecule-target polypeptide docking model is constrained by adding one or more attractive constraints, wherein the attractive constraint is for a residue having an enrichment score greater than a first preselected value.

25. The method of paragraph 24, wherein the first preselected value is between 20% and 40%, e.g., between 25% and 35%, e.g., about 30%.

26. The method of paragraph 24 or 25, wherein the attractive constraint comprises a linearly scaled bonus based on the enrichment score.

27. The method of any of the preceding paragraphs, wherein the antibody molecule-target polypeptide docking model is constrained by adding a repulsive constraint for a residue having an enrichment score less than a second preselected value.

28. The method of paragraph 27, wherein the second preselected value is between 5% and 20%, e.g., between 10% and 15%, e.g., about 12.5%.

29. The method of any of the preceding paragraphs, wherein step (d) comprises generating a docked pose between a model of the antibody molecule and a model of the target polypeptide.

30. The method of any of the preceding paragraphs, wherein step (d) comprises generating a plurality of docked poses between a model of the antibody molecule and a model of the target polypeptide.

31. The method of paragraph 30, wherein step (d) further comprises scoring the plurality of docked poses according to a docking algorithm, e.g., SnugDock.

32. The method of paragraph 31, wherein step (d) further comprises selecting a subset of the plurality of docked poses having the highest scores, e.g., the highest scoring 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000 or more docked poses.

33. The method of paragraph 32, wherein step (d) further comprises generating an ensemble docked pose using the selected subset of the plurality of docked poses, and setting the model of the antibody molecule and the model of the target polypeptide in accordance with the ensemble docked pose.

34. The method of any of paragraphs 29-33, wherein the model of the antibody molecule comprises an ensemble antibody homology model derived from a plurality of homology models of the antibody.

35. The method of any of the preceding paragraphs, wherein step (d) further comprises removing an antibody molecule-target polypeptide docketing model that exhibits a mode of engagement atypical for a known antibody-antigen complex, e.g., according to a structural filter derived from antibody-antigen crystal structure.

36. The method of any of the preceding paragraphs, wherein step (d) comprises generating a plurality of antibody molecule-target polypeptide models.

37. The method of any of the preceding paragraphs, wherein step (e) comprises identifying a plurality of sites on the target polypeptide that is capable of being bound by the antibody molecule.

38. A method of identifying an epitope on a target polypeptide, the method comprising:

(a) generating an antibody-target polypeptide docking model, wherein the antibody-target polypeptide docking model is constrained according to a plurality of enrichment scores determined by a method comprising:
(i) binding the antibody molecule to a plurality of variants of the target polypeptide,
(ii) obtaining (e.g., enriching) a plurality of variants exhibiting reduced binding to the antibody molecule, and
(iii) determining (e.g., calculating) enrichment scores for each of the plurality of the enriched variants; and

(b) identifying a site on the target polypeptide that is capable of being bound by the antibody molecule based on the antibody-target polypeptide docking model;

thereby identifying an epitope on a target polypeptide.

39. A method of identifying a paratope on an antibody molecule, the method comprising:

(a) binding the antibody molecule to a plurality of variants of the target polypeptide;

(b) obtaining (e.g., enriching) a plurality of variants exhibiting reduced binding to the antibody molecule;

(c) determining (e.g., calculating) enrichment scores for each of the plurality of the enriched variants;

(d) generating an antibody molecule-target polypeptide docking model, wherein the antibody-target polypeptide docking model is constrained according to the enrichment scores; and

(e) identifying one or more sites on the antibody molecule that is capable of being bound by the target polypeptide based on the antibody-target polypeptide docking model;

thereby identifying a paratope on an antibody molecule.

40. A method of identifying a paratope on an antibody, the method comprising:

(a) generating an antibody-target polypeptide docking model, wherein the antibody-target polypeptide docking model is constrained according to a plurality of enrichment scores determined (e.g., calculated) by a method comprising:
(i) binding the antibody to a plurality of variants of the target polypeptide,
(ii) obtaining (e.g., enriching) variants exhibiting reduced binding to the antibody molecule, and
(iii) determining (e.g., calculating) an enrichment score for each of the plurality of the obtained (e.g., enriched) variants; and

(b) identifying one or more sites on the antibody molecule that is capable of being bound by the target polypeptide based on the antibody-target polypeptide docking model;

thereby identifying a paratope on a target polypeptide.

41. An antibody molecule for which the epitope on a target polypeptide or the paratope on the antibody molecule for the target polypeptide is identified according to the method of any of the preceding paragraphs.

42. A nucleic acid molecule encoding one or more chains (e.g., VH and/or VL) of the antibody molecule of paragraph 41.

43. A vector comprising the nucleic acid molecule of paragraph 42.

44. A host cell comprising the nucleic acid molecule of paragraph 42 or the vector of paragraph 43.

45. A method of making an antibody molecule, comprising culturing the host cell of paragraph 44 under conditions suitable for expression of the antibody molecule.

### EXAMPLES

### Example 1: Computational Modeling of Antibody-Antigen Complexes Incorporating Conformational Epitope Mapping by Deep Sequencing of Comprehensive Antigen Libraries

To improve the quality of antibody-APRIL model structures, experimentally derived antigen (APRIL) mutational data was incorporated as constraints into a computational docking workflow. APRIL mutational profiles were derived from deep mutational scanning of an antigen library, which addressed the low-throughput nature of typical mutagenesis genotype-phenotype studies and enabled the simultaneous testing of thousands of mutational variants simultaneously for impact on binding. The throughput of the method enabled a more thorough sampling of surface residues and all mutations (i.e., not just Ala) and, therefore, provided a more sensitive and complete characterization of antigen residues contributing to antibody binding.

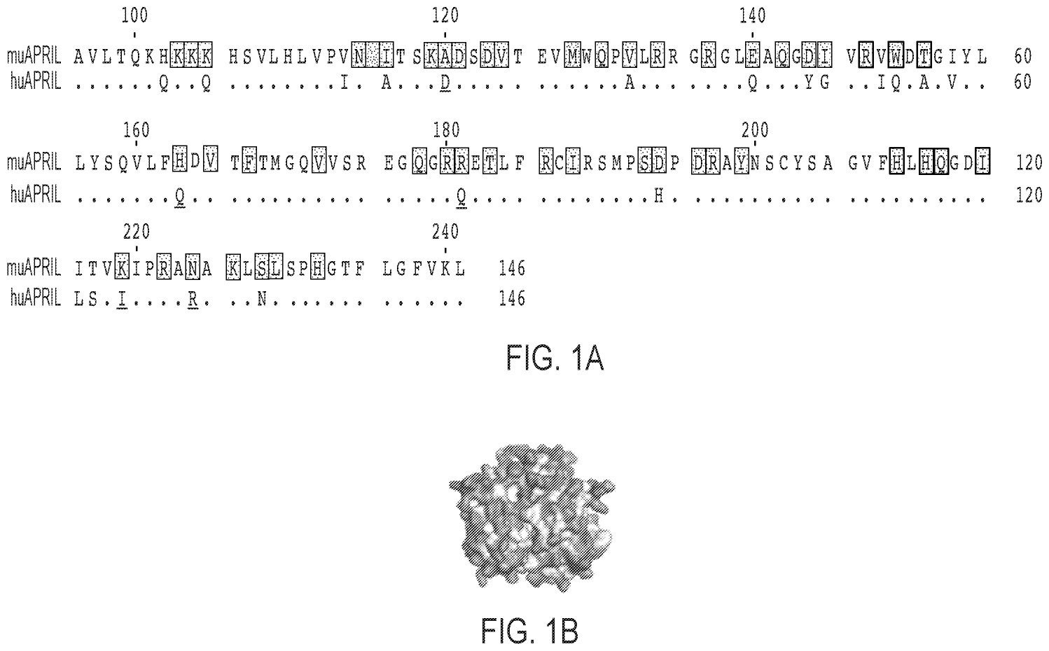

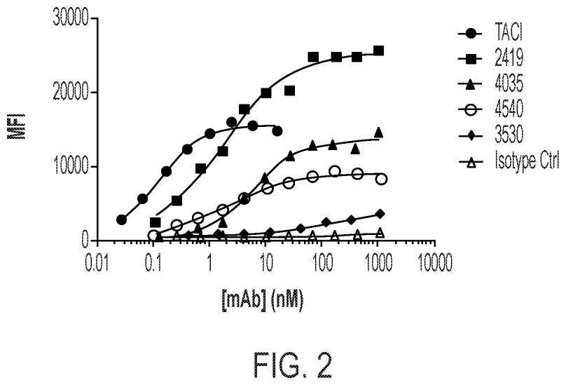

Yeast surface display was used to facilitate high-throughput screening of a comprehensive mutational library due to its ability to display conformationally intact antigen and ease of the system for library construction and selections. Productive expression of huAPRIL on the surface of yeast was found to be poor, in agreement with previous observations. Therefore, a chimeric form of mouse APRIL (muAPRIL) was designed, with surface residues in and surrounding the TACI-binding site mutated to the equivalent residues in huAPRIL (FIG. 1) to preserve the binding site for TACI and blocking antibodies. The resulting chimera is referred to herein as APRIL unless otherwise specified. All human-specific anti-APRIL antibodies and TACI were shown to bind to this designed APRIL (FIG. 2), demonstrating its conformational integrity.

An Aga2-APRIL fusion protein containing a 35-residue flexible linker (to facilitate multimerization) exhibited strong binding to TACI (FIG. 2). The binding site of TACI is composed of a quaternary structure, with significant contacts across the interface of two adjacent APRIL monomers. These binding results suggested the formation of a productive APRIL monomer-monomer interface on the surface of yeast.

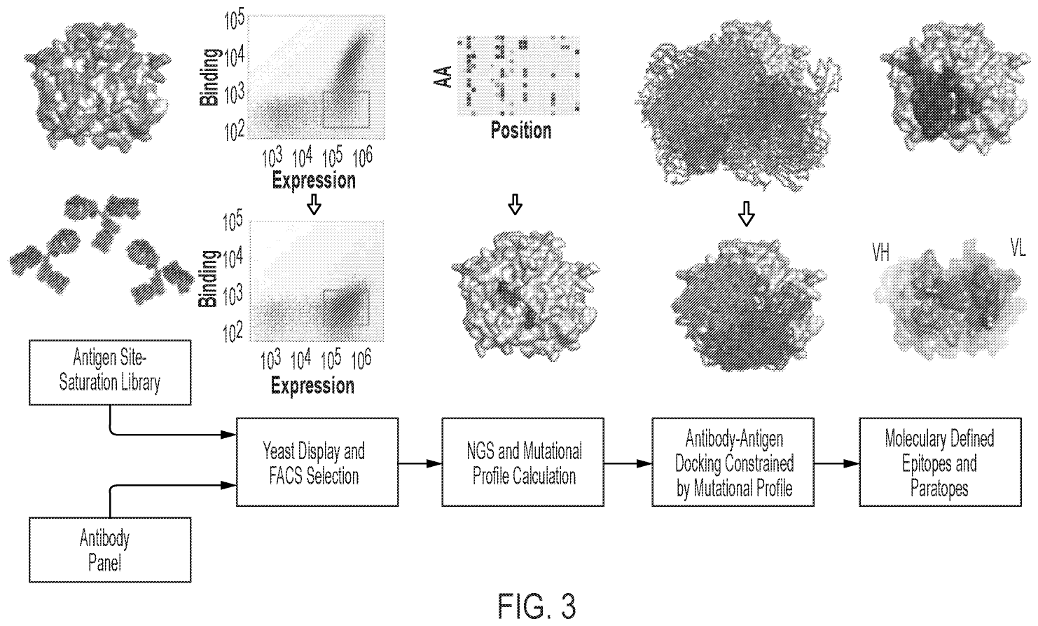

A panel of mouse-derived anti-huAPRIL antibodies was tested against APRIL expressed on yeast. All antibodies exhibited titratable binding (FIG. 2) consistent with their binding to purified, recombinant huAPRIL, further supporting structural integrity of the APRIL protein expressed on yeast surface. A yeast surface display library of site-saturation mutagenized surface positions of APRIL was screened against APRIL antibodies to generate comprehensive profiles of mutations affecting binding, and the results used to constrain computational antibody-antigen docking (FIG. 3).

### Example 2: Library Selections and Deep Sequencing

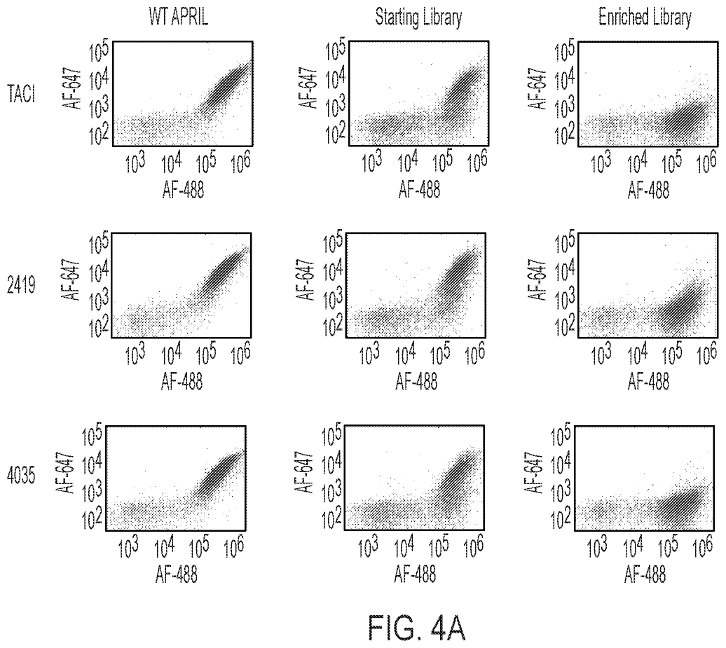

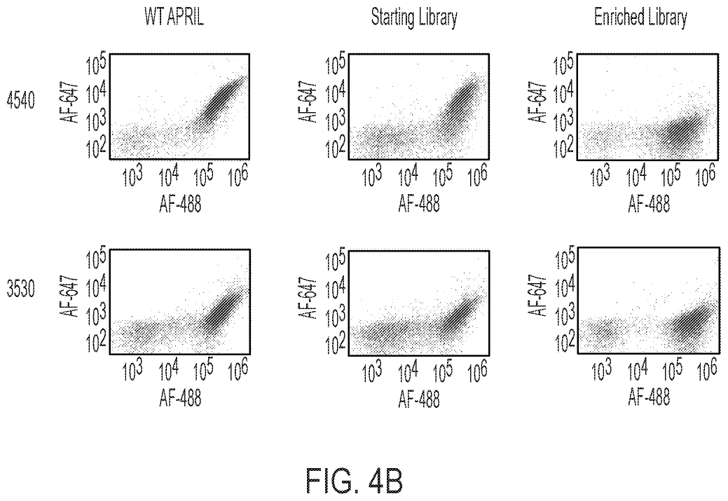

A single-site saturation mutagenesis library was synthesized using NNK degeneracy as described herein, and deep sequencing of the library confirmed the presence of all mutations at intended positions. The synthesized library was transformed into yeast, and yielded surface expression similar to unmutated APRIL. Binding studies using TACI and a panel of anti-APRIL antibodies revealed that most of the library retained strong binding, with a minority exhibiting reduced or no binding (FIGS. 4A-4B, first two columns). Two rounds of FACS enrichment of the expressing but non-binding population was performed (FIGS. 4A-4B, last column). The non-binding pools from the different binding experiments were then subjected to deep sequencing as described herein.

### Example 3: Generation of Mutational Profiles for Each Antibody

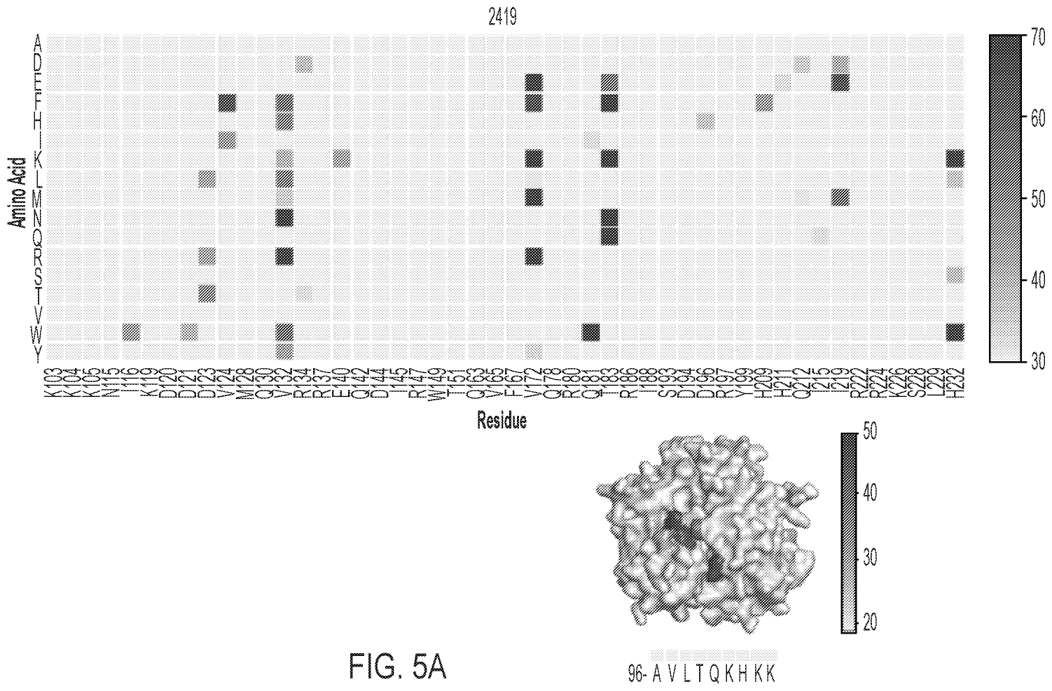

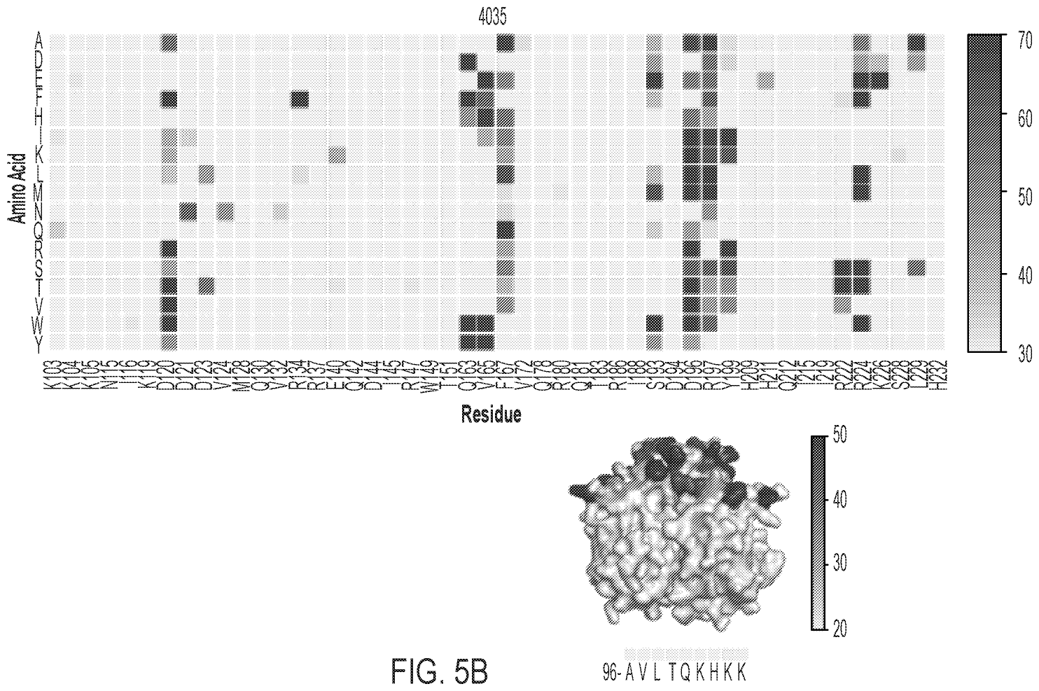

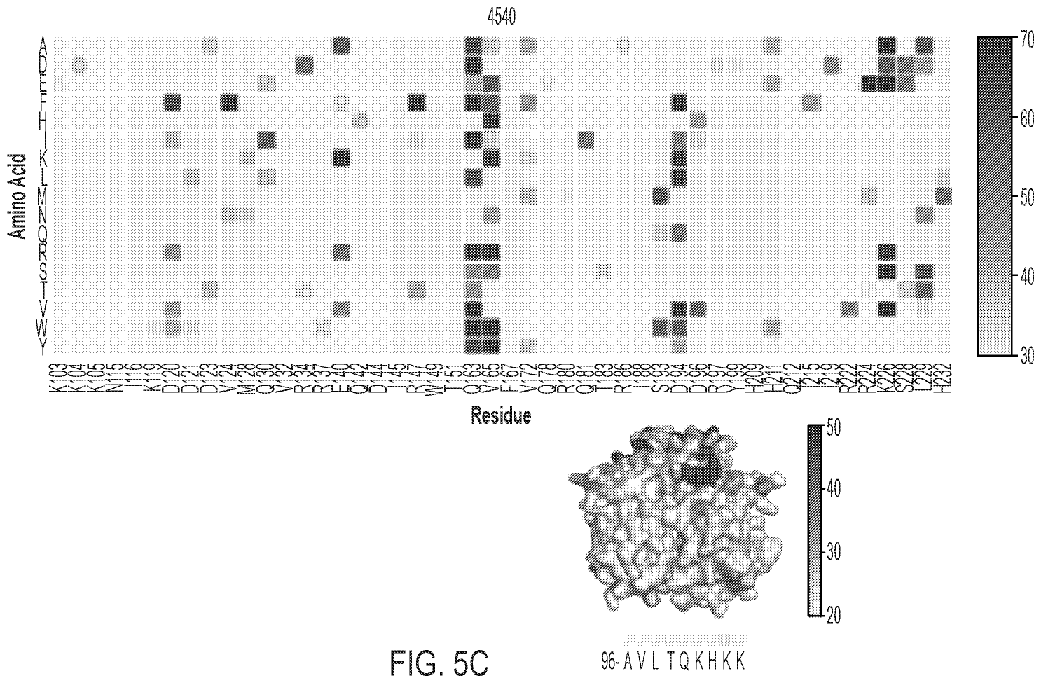

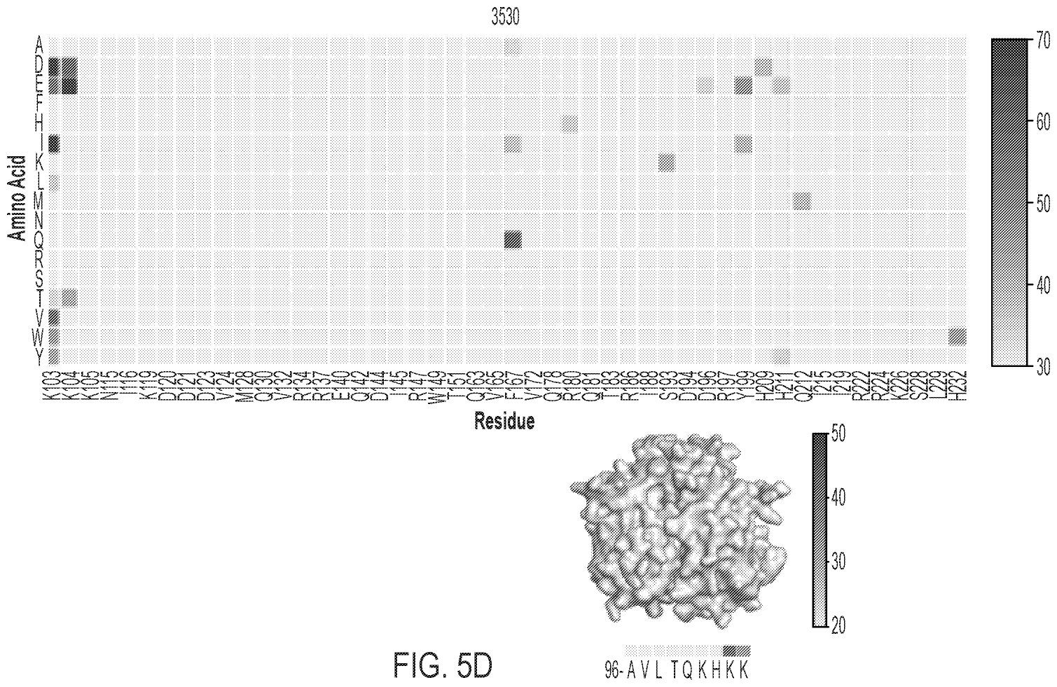

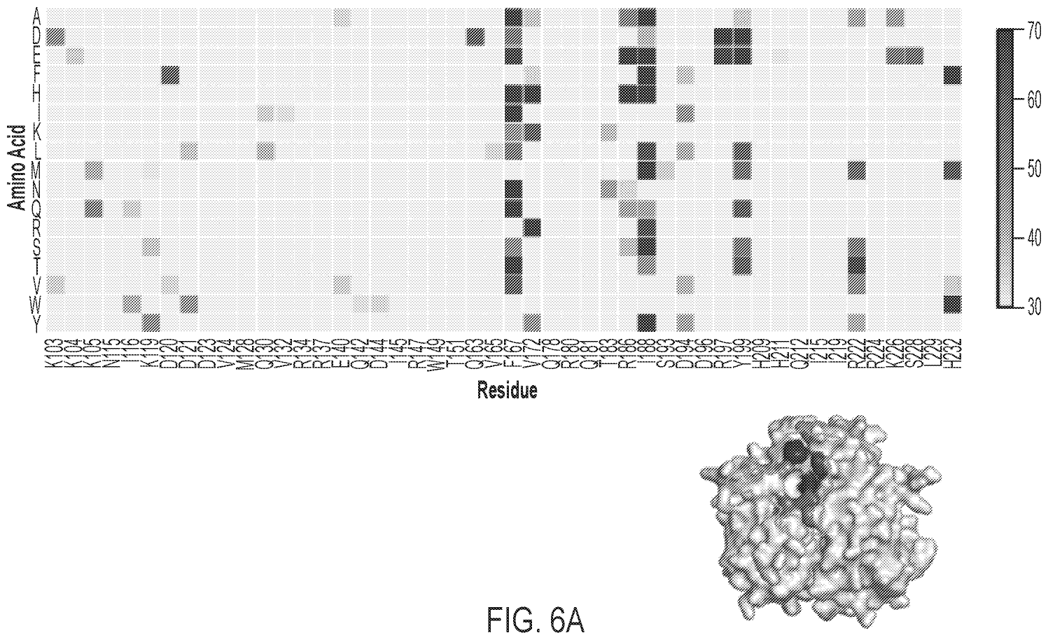

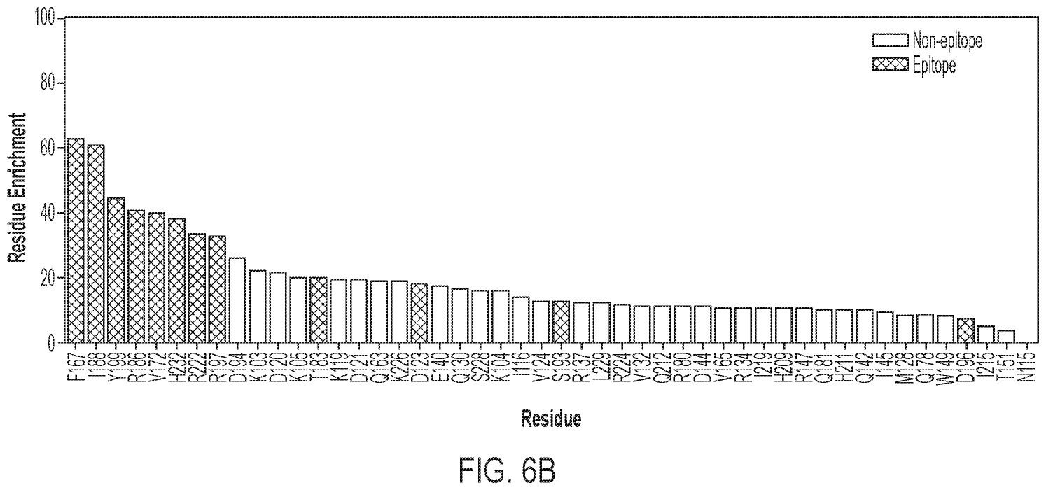

To generate a quantitative mutational profile for each antibody, bioinformatic analyses were performed to calculate the level of enrichment for every antigen variant against each antibody, as described herein. Variants enriched in the non-binding population relative to the starting library represented mutations that reduce antibody binding affinity. Two principal methods were deemed likely to cause reduced binding: direct effects, such as side-chains making direct contact with the antibody, and indirect effects, caused by change in local or global protein structure, not originating from mutation to a contact residue. The panel of characterized antibodies recognized different epitopes (determined using competition binding experiments, Table 2), which aided computational efforts in discerning mutations likely causing indirect effects on antibody binding through protein structure changes (i.e., affecting binding to most or all antibodies). Mutational profiles for all APRIL mutations queried were generated for all antibodies (FIGS. 5A-5D) and TACI (FIG. 6A).

TABLE 2
Results of antibody competition studies.

| | 2419 | 3530 | 4540 | 4035 |
|---|---|---|---|---|
| 2419 | + | − | + | − |
| 3530 | − | + | − | − |
| 4540 | + | − | + | + |
| 4035 | − | − | + | + |

(+) indicates that the two antibodies compete (>90% reduction in binding in competition ELISA).

Several APRIL mutations were observed that showed above-background enrichment scores across the majority of ligands. Given the non-overlapping epitopes of all antibodies determined from competition experiments (Table 2), this promiscuous effect on binding for many antibodies likely represented false positives caused by reduction in binding through indirect mechanisms. Thresholds for identifying promiscuous mutations for removal were determined based on inspection of enrichment maps for all samples (see supporting information).

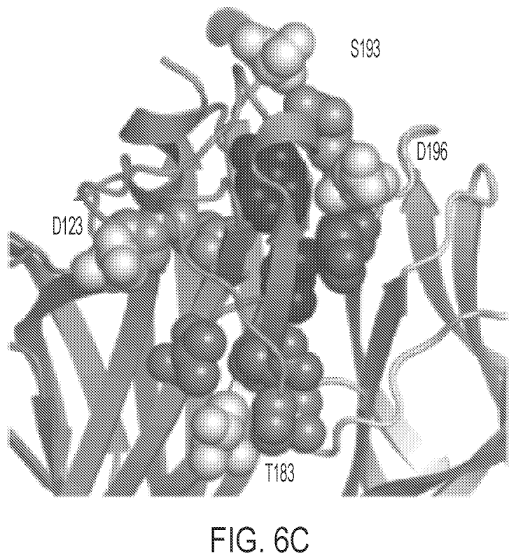

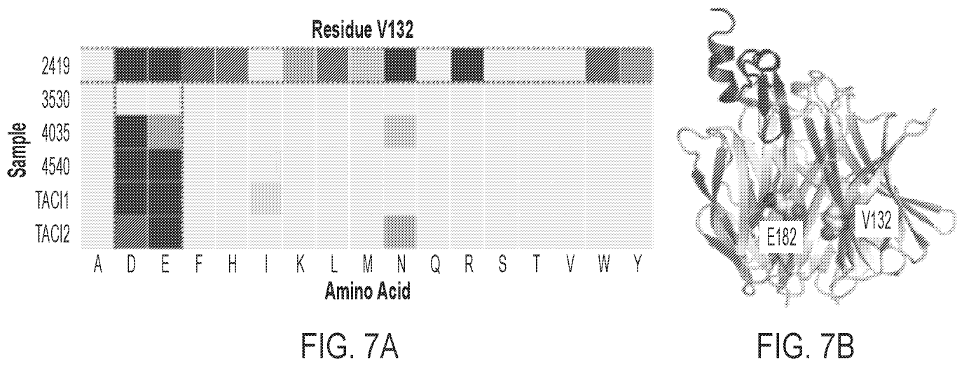

An illustrative example of a promiscuous mutation was observed for mutation of V132 to either Asp or Glu. These mutations resulted in high enrichment scores for all ligands (FIG. 7) other than 3530, including a significant impact on binding to both biological replicate samples of TACI. Structural analysis of TACI in complex with APRIL clearly showed that these residues were not in contact with TACI and would not be expected to cause a direct impact on binding. Notably, residue V132 was found at the interface between two monomers and was structurally adjacent to E182 on another monomer. Mutation at V132 to Asp or Glu may have resulted in an electrostatic repulsion with E182, destabilizing the quaternary structure of APRIL and thereby exerting an indirect impact on binding to the panel of ligands. Even though mutation at V132 to negatively charged residues ablates binding to most antibodies, mutation to a variety of other amino acids resulted in a reduction in binding that is specific for only antibody 2419 (FIG. 7). In this case, the mutants V132D and V132E were considered false positives, removed from further consideration, and not included in the calculation of total residue enrichment.

### Example 4: Analysis of Mutational Profiles

With the exception of 3530, all samples showed 2 to 6 positions for which mutation to most other amino acids disrupted binding (FIG. 5). As expected, some positions, such as R197 assessed against TACI binding (FIG. 6A), showed low enrichment scores for mutation to Ala but were sensitive to mutation to other amino acids, demonstrating the benefit of more thoroughly interrogating each position by site-saturation mutagenesis.

Mutational profiles for the control protein, TACI, were analyzed in the context of its known co-crystal structure with muAPRIL. Since the level of enrichment was expected to be related to the degree of impact on binding, this quantitative information was retained for analysis and structural visualization. Enrichment scores were mapped to the surface of APRIL for visualization and showed a well-defined patch composed of 8 residues with the highest residue enrichment scores centrally located in the epitope (FIG. 6B), in good agreement with the X-ray structure. These positions were found across the dimer interface of APRIL; residues F167, V172, R186, 1188, and R222 were found on one monomer, and R197, Y199, and H232 on the adjacent monomer, again demonstrating that APRIL expressed on the surface of yeast formed a productive monomer-monomer interface. Four residues found at the periphery of the epitope (T183, D123, S192, and E196) were shown to have enrichment scores indistinguishable from non-epitope residues (FIG. 6C), suggesting mutational tolerance at these positions. Overall, the mutational profile results for TACI closely matched the structural profile from the co-crystal structure data.

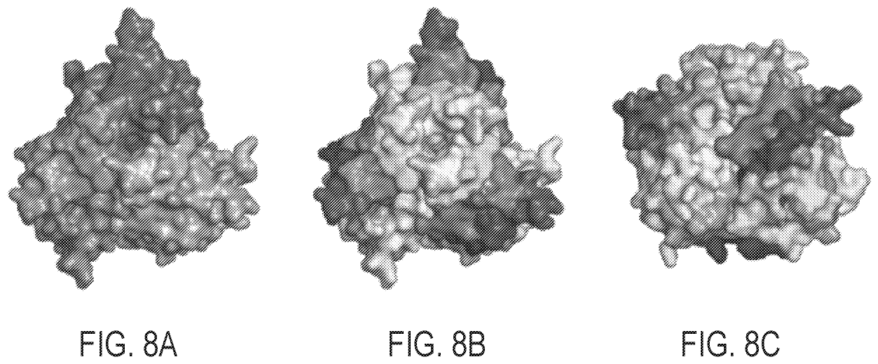

For each antibody, mutational profile data were visualized on the surface of APRIL (for all chains), and positions with high scores were also observed to cluster into surface patches, indicating likely epitope regions for each antibody (FIG. 5). Similar to TACI, epitope regions for antibody 2419, when visually inspected, showed surface patches formed by residues originating from different monomers across the dimer interface. When visualized on the surface, patches of high residue enrichment for antibodies 4035 and 4540 appeared larger and more dispersed than 2419. The difference in clarity of the maps was due, in part, to the symmetry and shape of the homo-oligomeric APRIL molecule. Equivalent residue positions on different APRIL monomers were in close proximity near the apex of the molecule (FIG. 8), making the patch for apex-binding molecules, like 4035, appear much larger.

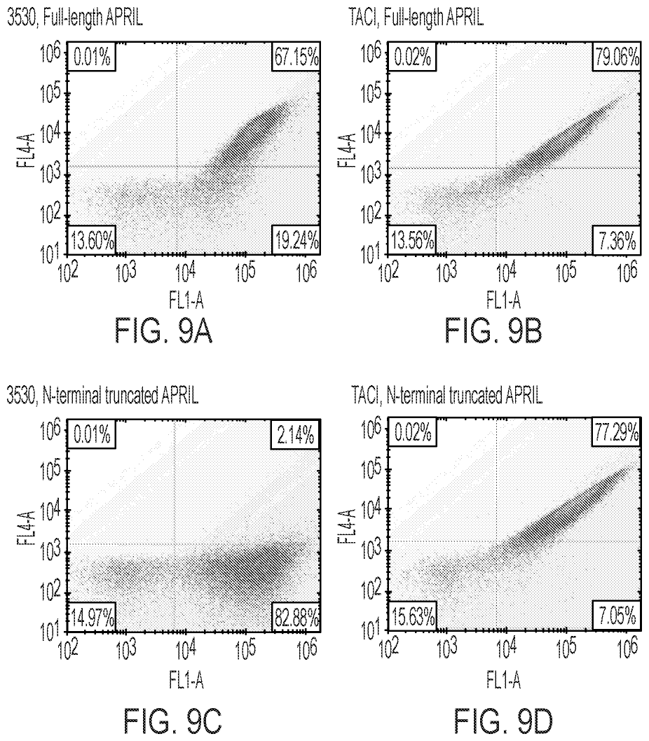

Consistent with antibody 3530 recognizing a linear epitope at the N-terminus of APRIL, only two residues in the N-terminus of APRIL showed high enrichment scores, both of which were not resolved in the X-ray structure of muAPRIL. This agreed with observations of antibody 3530 tested against the APRIL site-saturation library, which uniquely exhibited a very low percentage of non-binders, unlike for the other antibodies and TACI (FIG. 2). These results were corroborated by binding results to APRIL with deletion of the N-terminal peptide (FIGS. 9A-9D) and studies demonstrating lack of binding competition by 3530 to other antibodies (Table 2).

### Example 5: Computational Antibody-Antigen Docking

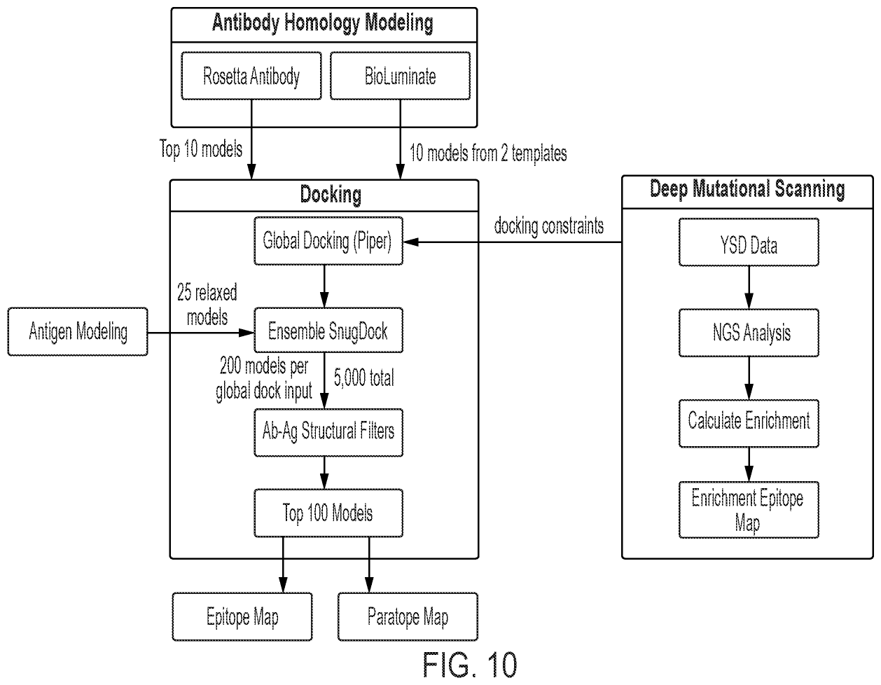

A multi-step docking approach was implemented to generate antibody-antigen models (FIG. 10). Global rigid-body docking was performed for each antibody against APRIL, using site constraints weighted proportionally to their experimentally-derived enrichment scores; this ensured that antibody-antigen poses were most favored when making maximal contact with high enrichment positions, while conversely disfavoring interactions with positions where binding was determined to be unaffected by mutation. The top ranked docked poses were then used as input to an ensembled-based local docking algorithm, SnugDock. The resulting top 100 ranked models were expected to be enriched in poses that were generally correct with regards to antibody-antigen orientation, and that could enable the identification of contact residues in the epitope and paratope, and to a lesser degree, the interacting pairs of epitope-paratope residues. A residue-based docking confidence score was calculated as the fraction of selected models where a residue was found making contact with the antibody or antigen.

### Example 6: Comparison of 2419 Docked Models with Crystal Structure

To validate the docking results, the co-crystal structure of 2419 with huAPRIL was solved. The single crystal structure of the Fab domain of 2419 in complex with huAPRIL (residues 115-250) was determined at 6.5 Å resolution. In the crystal structure, the Fab-APRIL complex formed a 3:3 molecular complex related by a non-crystallographic pseudo three-fold symmetry. The huAPRIL molecules formed a homotrimer that is similar to that found for muAPRIL (PDB: 1U5Y). Each Fab domain was bound across the homotrimer interface crosslinking two huAPRIL monomers. Due to low resolution, no clear electron density was observed for the side-chains of 2419 and huAPRIL; however, the structure of huAPRIL has been solved previously as a heterotrimer with BAFF at a high resolution (PDB: 4ZCH). The previously determined structure of huAPRIL fit the electron density of 2419-huAPRIL unambiguously and as such was used to model the complex, enabling the identification of huAPRIL epitope residues from the complex with high confidence. Based on the electron density map, the orientation of 2419 relative to huAPRIL was clear, permitting the elucidation of core paratope residues, although, due to greater uncertainty in the CDR regions, peripheral paratope residues could not be unambiguously defined. The CDRs of the VH and the VL domains were observed mostly bound to individual huAPRIL monomers across the homotrimer interface, with the VH occluding the binding-site for TACI.

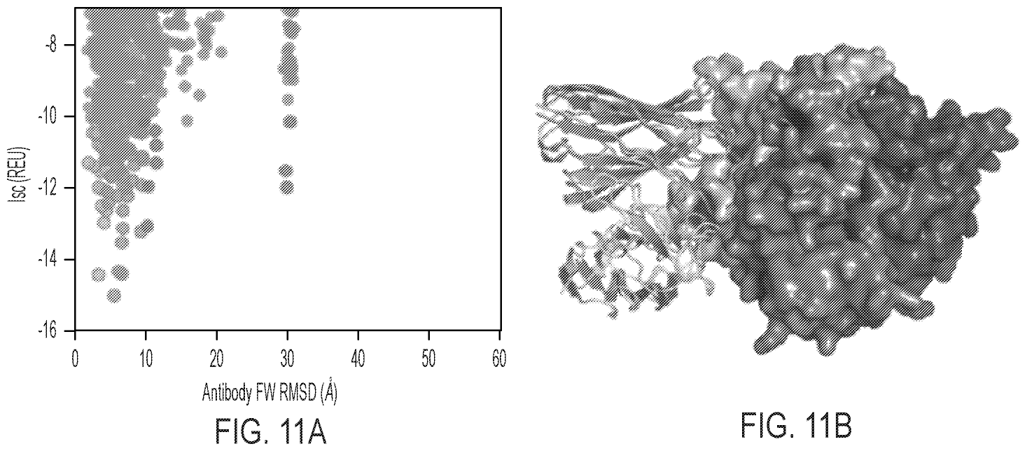

An analysis of the docking results for 2419 showed that the mode of engagement of docked models to APRIL was in strong agreement with the native structure. A large number of models were obtained which demonstrated near-native antibody-antigen orientations, with the large majority of models (90/100) having a low antibody ligand RMSD (L_rms)<10 Å, forming a clear binding energy funnel (FIG. 11A). Antibody ligand RMSD provided a stringent comparison of docked models to the native structure by superimposing only antigen coordinates, and subsequently assessing the RMSD over antibody framework backbone atoms. Using CAPRI-type rankings based on the antibody ligand RMSD, 27/100 models were considered medium quality (L_rms <5 Å), 63/100 were acceptable quality (L_rms between 5 Å and 10 Å) and 10 models were considered incorrect based on this single metric. The top ranked model is shown relative to the native structure (superimposed only on the antigen) in FIG. 11B, and good agreement in mode of engagement can be observed. For 2419, residues with high experimentally-derived enrichment scores also had high docking confidence scores (FIG. 11C), demonstrating that the majority of docking models made contact with those residues that showed the largest impact on binding upon mutation.

While the mode of engagement of docked models was similar to the native structure of 2419, the modeled HCDR3s did not adopt native-like conformations. For the canonical CDRs, the mean RMSDs, computed over the top 100 scoring models, were: H1: 1.17 Å, H2: 1.72 Å, L1: 1.57 Å, L2: 1.90 Å, and L3: 1.93 Å. However, for HCDR3, the mean RMSD was 6.17 Å. RMSD values for the top 10 scoring models are shown in Table 3.

TABLE 3
Observed Ca RMSDs (Å) for top 10 docked models of 2419. Antibody ligand
is the RMSD computed over the antibody framework residues after superimposing
on the antigen residues. RMSDs were computed for each of the six CDR loops
(Chothia definition) after superimposing based on the antibody framework residues.

| | Antibody | | | | | | |
|---|---|---|---|---|---|---|---|
| Model | ligand | HCDR1 | HCDR2 | HCDR3 | LCDR1 | LCDR2 | LCDR3 |
| model1 | 5.89 | 0.93 | 3.14 | 4.47 | 1.07 | 1.93 | 1.24 |
| model2 | 3.71 | 0.98 | 1.86 | 3.55 | 1.22 | 2.08 | 1.30 |
| model3 | 6.96 | 0.82 | 0.88 | 3.15 | 1.14 | 2.00 | 1.34 |
| model4 | 6.56 | 1.35 | 1.14 | 6.28 | 2.02 | 2.26 | 1.41 |
| model5 | 6.97 | 0.95 | 1.40 | 6.04 | 1.44 | 1.97 | 1.24 |
| model6 | 9.71 | 0.80 | 1.13 | 4.02 | 1.07 | 2.10 | 1.15 |
| model7 | 7.18 | 1.11 | 1.45 | 5.28 | 1.17 | 2.13 | 1.25 |
| model8 | 10.59 | 1.16 | 2.31 | 4.03 | 1.17 | 2.06 | 1.24 |
| model9 | 4.53 | 0.81 | 1.38 | 4.33 | 1.18 | 2.11 | 1.32 |
| model10 | 4.90 | 1.01 | 2.35 | 3.90 | 1.13 | 1.95 | 1.28 |

The HCDR3 for 2419 contains 11 residues (using Chothia numbering), and loops of this length are generally considered difficult to accurately model. Despite the challenge in accurately modeling the HCDR3 conformation for 2419, the inclusion of experimental data as constraints for modeling, derived only for the antigen, was sufficient to guide the docking workflow to identify near-correct contact of antibody and antigen interaction surfaces.

An analysis of the epitope determined from 2419 docked models showed surface patches that were much more detailed than those derived solely from experimental data. Out of the 22 contacting epitope residues determined from the native structure of 2419, 14 were mutated, but only 7 of these were found to have high enrichment scores (>20%) (FIG. 11C). In contrast, the top ranked docked model correctly identified 21 out of the 22 contacting residues on the epitope. Top ranked docked models could correctly identify epitope residues for 2419 (denoted by asterisks in FIG. 11C) even when those residues were not mutated or when they had low experimentally-determined enrichment scores.

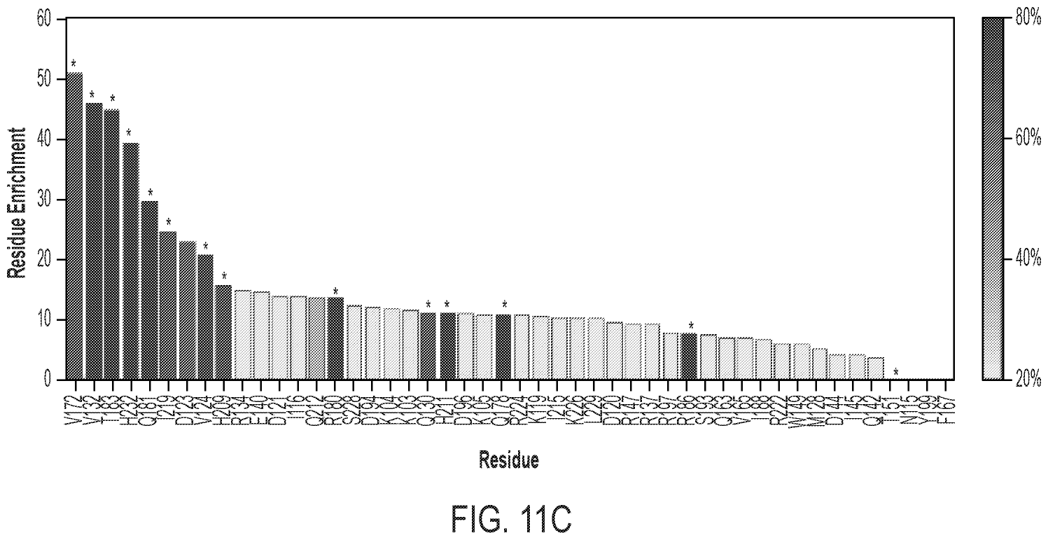

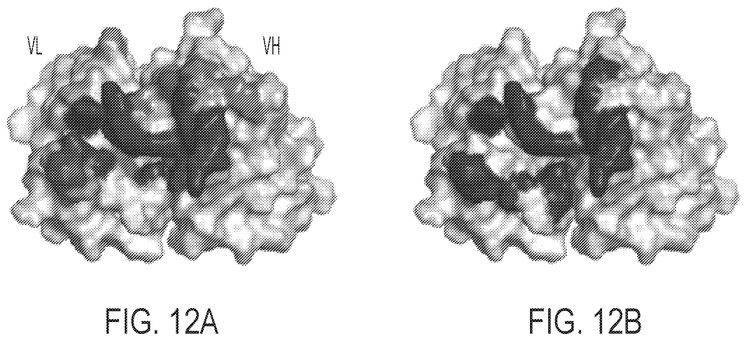

In addition to identifying the epitope residues consistent with the crystal structure, the docked models also provided valuable paratope information. Even though there were no experimentally determined constraints on the paratope, the paratopes determined from docked models were in good overall agreement with the low-resolution native structure (10 out of the 14 native paratope residues had docking confidence scores >50%) (FIGS. 12A-12B). In contrast to the determination of epitope residues, several false positives (3 residues having docking scores >50%) were identified where residues in the docked models were making contacts to the antigen not observed in the native structure. For 2419, these residues were found on the HCDR3 loop reflecting the errors in correctly modeling the conformation of this loop. By adopting incorrect conformations, HCDR3 residues in docked models can make contacts with the antigen not observed in the native structure. In some instances, errors in antibody homology modeling (including the HCDR3 remodeling in SnugDock), combined with a lack of explicit experimental constraints, may make the paratope mapping less accurate than the epitope mapping. Overall, there was good agreement between the predicted and actual paratope surfaces.

### Example 7: Impact of Constraints on Docking

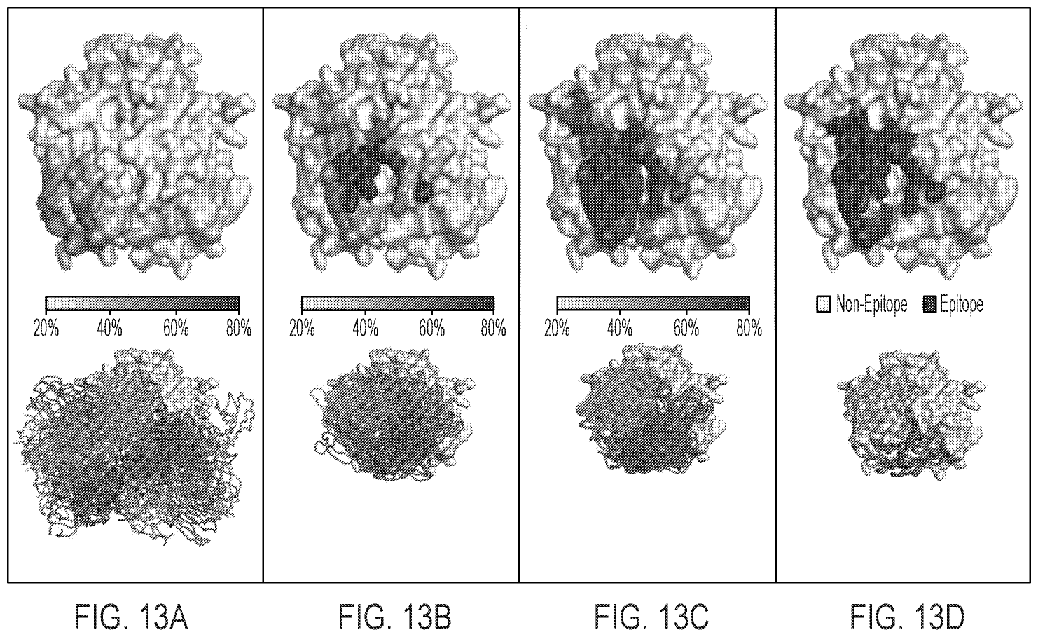

This computational workflow utilized a funneling approach to narrow in on models that were consistent with experimental data and therefore were more likely to be near-native poses (FIGS. 13A-13D). To assess the impact of incorporating constraints in the workflow, 2419 was used as an example to assess docking epitope results from top models generated by three different methods: (i) global docking without using experimental mutational profile data, (ii) global docking using mutational profile data, and (iii) the full docking workflow (including SnugDock and filtering based on antibody-antigen interface characteristics).

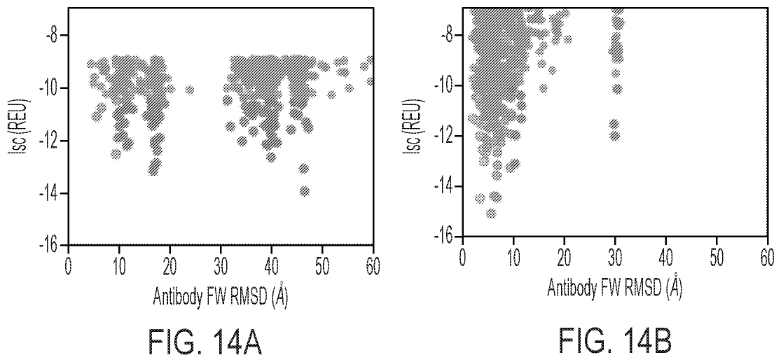

As expected, global docking without inclusion of experimentally derived constraints resulted in a large diversity of docked models. Here, most docked models predicted 2419 to bind somewhere near the base of APRIL in the visualized orientation, but there was very little consensus among models. This yielded a map (FIG. 13A) with low overall docking confidence scores, and which bore little similarity to the actual epitope for 2419 (FIG. 13D). Including mutational profile data in the global docking procedure resulted in a larger number of overlapping poses focused near the true epitope, but a large variation in the relative binding orientations was still observed (FIG. 13B). The use of the full docking workflow, including an ensemble local docking component (SnugDock) resulted in a tight cluster of near-native poses (FIG. 13C) and an epitope map that was very similar to that derived from the crystal structure. Including experimentally derived mutational profile data resulted in a clear docking funnel of near-native structures, whereas performing the docking workflow without constraints resulted in a much higher number of non-native models (FIGS. 14A-14B). This result showed that incorporation of the mutational profile data could overcome deficiencies in computational docking scoring methods in selecting near-native models.

### Example 8: Analysis of Docked Models Reveal Mechanistic Insights

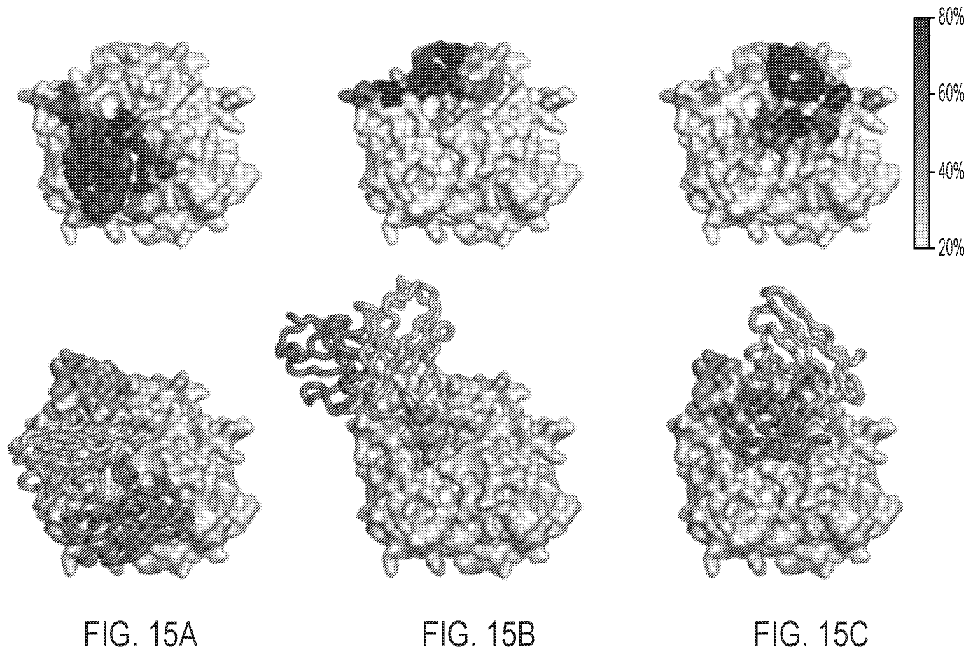

Docked models for all 3 antibodies indicated their mode of engagement to APRIL and the manner in which they block TACI binding (FIGS. 15A-15C). 2419 bound across a dimer interface, with its heavy chain binding to an equatorial region of APRIL and thereby occluding the TACI binding site. 4035 bound near the apex of APRIL, and its heavy chain exhibited substantial interactions with the TACI binding site. For 4540, docked models suggested that it was primarily the light chain that occluded the TACI binding site. Docked models of all 3 antibodies revealed distinct epitopes for each antibody, and the overlap of epitopes was consistent with competition binding data which showed that 4540 competes with both 2419 and 4035, while 2419 did not compete with 4035 (Table 2). Visual inspection of top docked models showed that all antibodies can engage APRIL in a manner which was consistent with a 3:3 binding ratio of antibody, thereby blocking the TACI binding site on all 3 monomers of the APRIL homotrimer.

### Example 9: Application to Antibody Engineering

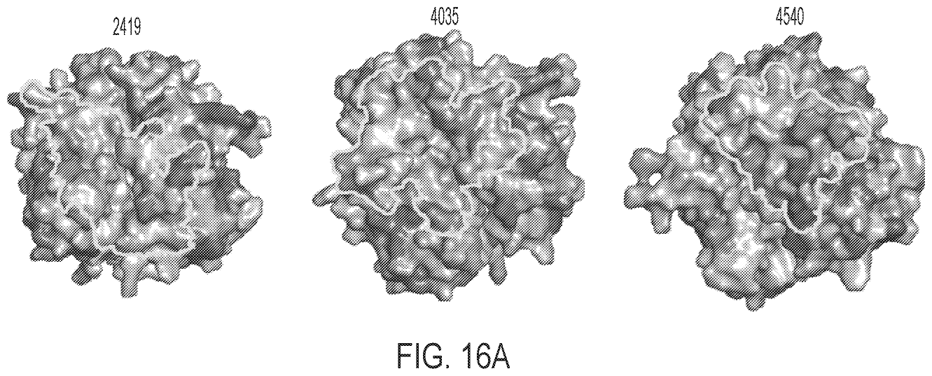

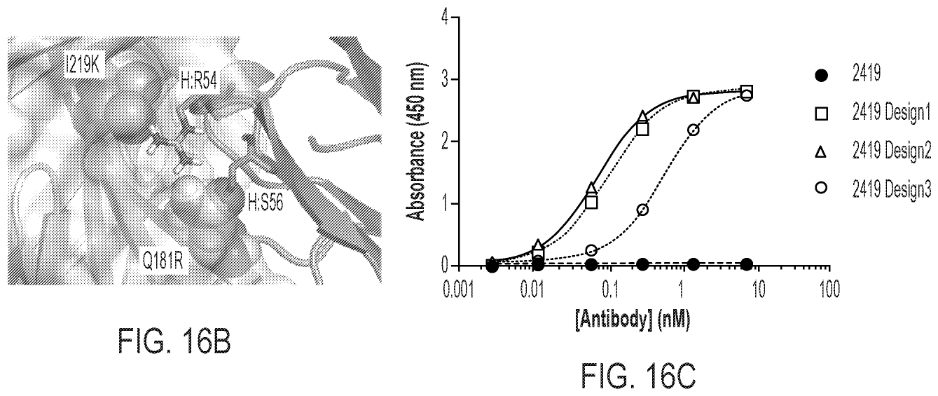

For therapeutic antibody development, cross-reactive binding to both rodent and human species can be desirable to facilitate more convenient efficacy and PK/PD testing in rodent models. The modeling results were thus used to enable rational engineering to improve cross-species reactivity, as an illustration of the utility and accuracy of the molecularly defined epitopes and paratopes. muAPRIL and huAPRIL share 85.6% sequence identity (FIG. 1), and the sequence differences were visualized on the structure of muAPRIL and analyzed in the context of docking confidence maps generated for each antibody obtained using the modeling workflow. The fewest non-conservative mutations were found in the epitope patch for 2419. In contrast to the other antibodies, these mutations were found at the periphery of the 2419 epitope (FIG. 16A). Non-conservative mutations, which result in dramatic differences in amino acid size, charge, or hydrophobicity, would be expected to have a greater impact on antibody binding.

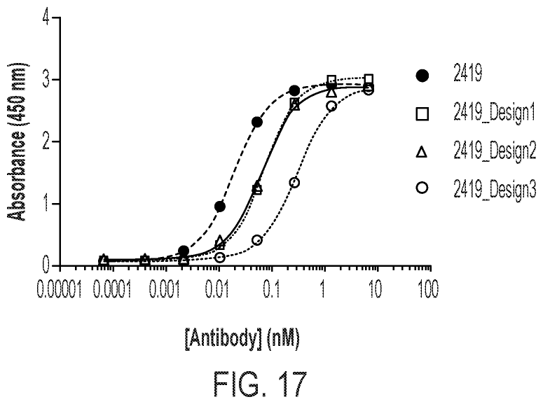

Visual inspection of the APRIL-2419 interface residues in top model complexes showed that the two non-conservative human-to-mouse mutations, Q181R and I219K, were proximal to R54 on the heavy chain of 2419 (FIG. 16B). It was hypothesized that the presence of two positively charged residues at positions 181 and 219 in muAPRIL would lead to electrostatic repulsion as well as potential steric clashes with Arg54 on the HCDR2 of 2419 and may be a major determinant for the lack of 2419 binding to muAPRIL. Mutation of R54 to Asp on HCDR2 was predicted to form a favorable interaction with the positive charges at R181 and K219 in muAPRIL, while not significantly impacting binding to human residues Q181 and 1219. Additionally, several other mutations to 2419 were nominated to be combined with R54D, in which residues were mutated to smaller amino acids (T28A, L53V, and S56A) to alleviate any potential steric clashes that may result from the presence of the larger side-chains at positions 181 and 219 in muAPRIL. Experimental results for these mutations showed that all 3 designed variants of 2419 exhibited substantial binding to muAPRIL (FIG. 16C) with only minor impact on binding to huAPRIL (FIG. 17). These results showed that the workflow generated antibody-antigen structural models of sufficient quality to facilitate structure-guided antibody redesign.

### Example 10: Materials and Methods

### Selection of APRIL Mutant Positions

Briefly, using the structure of homotrimeric mouse APRIL (PDB: 1XU1) as a guide, an initial set of surface residues was chosen by selecting residues with relative side-chain surface accessibility >25% and ensuring even surface coverage of positions on the protein surface. Forty-six surface positions resolved in the structure were chosen, and an additional two residues at the N-terminus of the protein that were not resolved were selected for mutational interrogation (highlighted on the sequence and structure of APRIL in FIG. 1). A site-saturation library was designed and synthesized (IDT), using an NNK degenerate codon at each position to be varied.

### Yeast Library Construction and FACS Selections

Yeast surface display was performed as previously described. Briefly, a chimeric APRIL gene was designed using mouse sequence (residues 96-241) with 5 positions in and around the TACI-binding site mutated to the amino acid found in the human APRIL (huAPRIL) gene (A120D, H163Q, R181Q, K219I, N224R) (see also FIG. 1A). A synthesized degenerate (NNK) library of the APRIL gene was PCR-amplified and co-transformed with linearized expression vector into EBY100 yeast and cultured as previously described. Yeast expressing the APRIL library were exposed to antibody at a concentration corresponding to 80% maximal binding, stained with fluorescent antibodies to the test antibody and to yeast APRIL surface expression tag Myc, and sorted using a BD FACSAria. Yeast exhibiting cMyc expression and with binding lower than that to non-mutated APRIL were selected. Two rounds of FACS were performed, and the APRIL gene of enriched libraries were PCR amplified and sequenced by Illumina MiSeq 2×75 PE (Genewiz).

### Next Generation Sequencing (NGS) Analysis

Briefly, high quality reads were assembled, selecting those that contained a single amino acid change relative to the template gene (APRIL) for further analysis. An enrichment score for each mutation was calculated in a manner similar to that previously described, representing the fraction of a mutation from the expresser pool that is found in the non-binding pool after FACS.

High-quality reads were aligned to the template gene (APRIL), removing reads containing N's, indels, and those with >10 base substitutions. Nucleotide reads were converted to amino acid reads, removing those that contained stop codons, mutations at unintended positions, or more than one amino acid substitution relative to the template gene. Forward and reverse amino acid reads were combined, and combined reads were removed if more than 1 substitution was observed, or if the sequence on overlapping regions were not in agreement. The median count for each mutation in each sample was 1,845, with a range from 453 (5^{th} percentile) to 7,760 (95^{th} percentile). Mutations where less than 100 reads were observed were removed from consideration. An enrichment score for each mutation was calculated in a manner similar to that previously described; for each sample collected in a non-binding pool, the position-dependent frequency of occurrence of a mutation in a sample is normalized by the frequency of occurrence of that mutation in the expresser pool, and scaled by the fraction of variants found in the non-binding pool as follows:

$${E}_{p , aa}^{s} = N {B}^{s} ( \frac{{f}_{p , aa}^{s}}{{f}_{p , aa}^{wt}} )$$

Where E_{p,aa}^{s} is the enrichment score for a given amino acid (aa) at positon (p) for sample (s), NB^{s} is the fraction (pool size) of variants found in the non-binding pool, and f_{p,aa} is the observed positional frequency of the amino acid from either the non-binding pool for a sample (s) or the expresser pool (wt). The enrichment score, therefore, represents the fraction of a mutation from the expresser pool that is found in the non-binding pool after FACS (represented here as a percentage).

Mutations to Pro, Gly, or Cys were removed from further analysis, as were mutations that were predicted to introduce or remove N-glycosylation sites. Mutations which were observed to impact the binding of a large majority of proteins were removed, as these are more likely to be exerting their effect through an indirect effect such as alteration of tertiary or quaternary structure. A total enrichment score was calculated for each residue by aggregating the effect of each mutation at the corresponding position. Residues with higher enrichment scores reflected greater sensitivity to mutation with respect to binding, indicating that a position is more likely to be part of the epitope. For this study, mutations where more than 50% of samples had an enrichment score >30.0% and where more than 75% of samples had an enrichment score >15.0% were removed from further analysis (“promiscuous effects”, global impact on protein folding), resulting in removal of 68 out of the possible 816 mutations in this study.

While enrichment scores were calculated for a multitude of single point mutants, the aggregate of mutational data for each position must be considered when determining whether a residue is part of the epitope. A total enrichment score was calculated for each residue by aggregating the effect of each mutation at the corresponding position. Enrichment scores were calculated as follows:

$${E}_{p}^{s} = \sqrt{\frac{{\sum }_{i}^{{N}_{p , aa}} {( {E}_{p , aa}^{s} )}^{2}}{{N}_{p , aa}}}$$

Where N_{p,aa} is the number of amino acid mutations at a given position after filtering. Rather than a simple summation of enrichment scores for each mutation, the calculated total residue enrichment score more heavily weights the effect of mutations that showed a large enrichment score and down-weights the contributions from mutations that showed low enrichment scores. Once calculated for each position, residue enrichment scores were mapped onto protein surfaces to facilitate analysis by visualization.

### Antibody and APRIL Homology Modeling

Ten structurally diverse antibody homology models were selected from 2,800 models generated using the most recently described Rosetta antibody homology modeling protocol (implemented in Rosetta 3.8) following the guidance described in the published protocol for selection of models. Homology models were also generated using BioLuminate's (Schrödinger Release 2016-4: BioLuminate, Schrödinger, LLC, New York, N.Y., 2016) antibody homology modeling protocol using the default settings. Five models were generated for each of the 2 top-ranked non-homologous structural templates; the templates for 2419 were 3DGG and 3S35, for 4035 were 1FLD and 4EDW, and for 4540 were 2E27 and 5AZ2.

Homology models for the antigen, APRIL, were generated with Rosetta using the structure of muAPRIL (PDB: 1XU1) as a template. The fixbb design protocol was used to introduce the 5 mutations present in APRIL relative to muAPRIL, ensuring that appropriate mutations were made at each of the chains in the homotrimer. An ensemble of antigen structures was then generated using the relax protocol implemented in Rosetta, selecting the 25 lowest scoring models from 100 relaxed structures based on their Rosetta total score.

Global Docking with Constraints

Briefly, global rigid-body docking was performed using PIPER, as implemented in BioLuminate using the default settings and incorporating higher confidence enrichment scores as site-constraints. Attractive constraints were added for sites where a substantial impact on binding was observed upon mutation (defined here as residue enrichment scores >30.0%), and repulsive constraints were added for sites that minimally impacted binding upon mutation (defined here as residue enrichment scores <12.5%). Global docking was performed for each of the 20 input antibody homology models, generating a total of 600 docked poses (30 poses representing cluster centers are obtained for each sample).

An “epitope score” was calculated to assess the level of agreement between each docked model and the experimentally determined enrichment scores using the following equation:

$$ES = {\sum }_{p = 1}^{N} {c}_{p}$$

$${c}_{p} = \{ \begin{matrix}{E}_{p} - 30 , if {E}_{p} > 30 \\ 12.5 - {E}_{p} , if {E}_{p} < 1 \; 2.5\end{matrix}$$

Where ES is the epitope score, N is the number of sites with constraints, cₚ is the constraint at position p, and Eₚ is the experimentally-derived enrichment score at position p (calculated as previously described). For each antibody, the 600 docked models were ranked by the epitope score, and the top 25 models were selected as starting templates for further local docking.
Local Docking with SnugDock Following global docking, local docking was carried out using Ensemble SnugDock (implemented in Rosetta 3.8) using the most recently described protocol. The 20 antibody homology models were used as the ensemble of antibody structures. Homology models generated by BioLuminate were first relaxed using Rosetta to ensure that all models were generated by, and consistent with, the same forcefield. The top 25 globally docked poses were used as starting input coordinates for Ensemble SnugDock, and 200 docked models were generated for each input, resulting in a total of 5,000 docked models. As with PIPER, docking constraints were utilized for SnugDock based on enrichment scores. To account for the symmetry of the homotrimer, Rosetta ambiguous site constraints (using a sigmoidal function) were applied to antigen residues to allow them to originate from any monomer of APRIL. The set of residues constrained in local docking was equivalent to that constrained in global docking.

Docked poses generated by SnugDock were filtered to remove models that had interfaces that are atypical of antibody-antigen interfaces. A non-redundant database of publicly available antibody-protein complexes was obtained,⁴ and curated to remove structures with missing regions near the interface, or complexes with ligands or post-translational modifications at the interface. For the resulting 297 complexes, distributions of structural features for key interface properties were calculated, including the number of CDR and framework residues engaging the epitope, the number and type of CDR loops involved in interactions, the number of epitope residues, the buried surface area, and pairwise residue propensities (Table 1). Appropriate thresholds were empirically chosen so that 95.2% of native structures failed no more than one of the structural filters. The calculated filters and their thresholds are listed in Table 1. Interface properties were calculated for each of the docked models, and those models that failed more than one of the structural filters were removed. Remaining docked models were filtered based on the epitope map score (as described for global docking). Since residues on the periphery of the epitope may be expected to be more tolerant to mutation, docked models were allowed to make contact with a small number of residues with low enrichment scores; here we removed models with enrichment scores <80% of the maximum observed epitope map. Remaining docked models were ranked based on the interface energy (Isc) as calculated using Rosetta.

### Competition ELISA

Biotinylated test antibodies (fixed at 50 ng/mL) and an unlabeled competing antibody (8-point serial dilutions starting at 10,000 ng/mL) were transferred to wells pre-coated with human APRIL at 0.1 μg/well. Plates were washed and streptavidin-horseradish peroxidase was added followed by washing and development using 3,3′,5,5′-tetramethylbenzidin substrate. Observations of partial or complete reduction in the binding of the biotinylated test antibody indicated competition between the antibodies for binding to overlapping or neighboring epitopes. Antibodies were classified as “non competing” if unable to block >90% of the binding signal even when present at a 200× molar excess to the test antibody (10,000 vs. 50 ng/ml).

### Preparation, Crystallization, and Structure Determination

Human APRIL (residues 105-250, (His)₆ epitope tag)) and mouse antibody 2419 were recombinantly expressed in Expi293 cells and purified using nickel or protein A affinity chromatography, respectively. The Fab fragment of 2419 was generated by papain digestion. APRIL and Fab formed a 3:3 complex in solution (as determined by size exclusion chromatography) and the complex was purified. Diffraction quality crystals were obtained using 2.2 M ammonium sulfate, 160 mM ammonium nitrate, 4% ethylene glycol and 1 mM NiCl₂ as precipitant. Most crystals diffracted to only up to 7 Å resolution and a complete X-ray diffraction data set was collected from a crystal at 100K using 20-36% ethylene glycol as cryo-protectant (Table 4).

TABLE 4
X-ray data collection and refinement parameters.

| X-ray beam wavelength | 0.9793 Å |
|---|---|
| Space group | P4₁2₁2 |
| Unit cell parameters | a = b = 209.60 Å |
| | c = 110.64 Å |
| | α = β = γ = 90° |
| resolution (Å) | 75-6.5 |
| | (6.73-6.5)^{†} |
| measured reflections | 52669 |
| unique reflections | 5176 |
| R_{sym} (%) | 16.7 (56)^{†} |
| completeness (%) | 98.3 (96.8)^{†} |
| I/σ | 9.7 (3.0)^{†} |
| Redundancy | 10.2 (9.3)^{†} |

Refinement parameters

| | |
|---|---|
| Resolution range (Å) | 148.2-6.5 |
| Rcryst/Rfree (%) | 29.5/36.8 |
| Bond lengths, rms (Å) | 0.009 |
| Bond angles, rms (°) | 1.212 |
| Ramachandran plot (%) | |
| preferred | 80.8 |
| allowed | 18.8 |
| outliers | 0.4 |

^{†}Highest resolution shell.

A self-rotation function suggested the presence of a pseudo three-fold symmetry confirming that the 3:3 APRIL-Fab complex is related by this pseudo three-fold symmetry. The structure was solved by molecular replacement using a homotrimer APRIL model generated based on the mouse APRIL homotrimer crystal structure (PDB 1USY) along with the Fab structure yielding a unique structure solution containing three Fab molecules bound to the APRIL homotrimer. The final refinement statistics are shown in Table 4.

Other exemplary methods are described in Wollacott et al., J Mol Recognit. 2019; 32(7): e2778, the contents of which is incorporated by reference in its entirety.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A method of identifying an epitope on a target polypeptide, the method comprising:
(a) binding an antibody molecule to a plurality of variants of the target polypeptide;
(b) obtaining (e.g., enriching) a plurality of variants exhibiting reduced binding (e.g., reduced binding affinity) to the antibody molecule;
(c) determining (e.g., calculating) an enrichment score for each of the plurality of the obtained (e.g., enriched) variants;
(d) generating an antibody molecule-target polypeptide docking model, wherein the antibody molecule-target polypeptide docking model is constrained according to the enrichment scores; and
(e) identifying a site on the target polypeptide that is capable of being bound by the antibody molecule based on the antibody molecule-target polypeptide docking model;
thereby identifying an epitope on a target polypeptide.

2. The method of claim 1, wherein step (a) comprises binding the antibody molecule to a library displaying a plurality of variants of the target polypeptide.

3. The method of claim 1 or 2, wherein step (a) comprises binding the antibody molecule to a library comprising a plurality of cells expressing (e.g., displaying) a plurality of variants of the target polypeptide.

4. The method of claim 3, wherein each of the plurality of cells expresses about one distinct variant of the target polypeptide.

5. The method of claim 3 or 4, wherein the cell is a eukaryotic cell, e.g., a yeast cell.

6. The method of any of the preceding claims, wherein the plurality of variants comprise mutations on one or more surface residues of the target polypeptide.

7. The method of any of the preceding claims, wherein the plurality of variants comprise distinct mutations of a selected surface residue of the target polypeptide.

8. The method of any of the preceding claims, wherein the plurality of variants comprise distinct mutations of each of a plurality of selected surface residues of the target polypeptide.

9. The method of any of the preceding claims, wherein the plurality of variants comprise single amino acid substitutions, relative to a wild-type amino acid sequence of the target polypeptide.

10. The method of any of the preceding claims, wherein each of the plurality of variants comprises a single amino acid substitution relative to a wild-type amino acid sequence of the target polypeptide.

11. The method of claim 9 or 10, wherein the single amino acid substitution occurs at a surface residue of the target polypeptide.

12. The method of any of the preceding claims, wherein the reduced binding comprises a reduction of binding detected for the variant and the antibody molecule, relative to the binding detected for a wild-type target polypeptide and the antibody.

13. The method of any of the preceding claims, wherein step (b) comprises obtaining (e.g., enriching) variants exhibiting less than about 80% (e.g., less than about 0.01%, 0.1%, 1%, 2%, 3%, 4%, 5%, 6%, 7%, 8%, 9%, 10%, 20%, 30%, 40%, 50%, 60%, 70%, or 80%) of the binding to the antibody molecule exhibited by a wild-type target polypeptide.

14. The method of claim 13, wherein the reduced binding is at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by the wild-type target polypeptide.

15. The method of any of the preceding claims, wherein step (b) comprises obtaining (e.g., enriching) cells exhibiting less than about 80% (e.g., less than about 0.01%, 0.1%, 1%, 2%, 3%, 4%, 5%, 6%, 7%, 8%, 9%, 10%, 20%, 30%, 40%, 50%, 60%, 70%, or 80%) of the binding to the antibody molecule exhibited by a cell comprising a wild-type target polypeptide.

16. The method of claim 15, wherein the reduced binding is at least about 20% (e.g., at least about 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) of the binding exhibited by a cell comprising the wild-type target polypeptide.

17. The method of any of the preceding claims, wherein step (b) comprises performing one or more, e.g., two, three, four, five, six, seven, eight, nine, ten, or more, enrichments for variants exhibiting reduced binding to the antibody molecule.

18. The method of any of the preceding claims, further comprising, e.g., prior to step (c), identifying the variants exhibiting reduced binding to the antibody molecule, e.g., by sequencing the genes encoding the variants, e.g., by next-generation sequencing.

19. The method of any of the preceding claims, wherein step (c) comprises determining the frequency of occurrence for each of the plurality of the obtained (e.g., enriched) variants.

20. The method of claim 19, wherein step (c) further comprises aggregating the frequency of occurrence of each variant comprising a distinct mutation at a particular residue and/or heavily weighting variants with higher frequencies of occurrence.

21. The method of any of the preceding claims, wherein the enrichment score is specific to a single residue of the amino acid sequence of the target polypeptide.

22. The method of any of the preceding claims, wherein each enrichment score is specific to a different single residue of the amino acid sequence of the target polypeptide.

23. The method of any of the preceding claims, further comprising repeating steps (a)-(c) at least once (e.g., once, twice, three times, four times, five times, or more) with replicates of the plurality of the variants of the target polypeptide, and wherein step (c) further comprises omitting one or more promiscuous mutations, e.g., mutations for which more than 50% of replicates had an enrichment score of greater than 30% and for which more than 75% of replicates had an enrichment score greater than 15%.

24. The method of any of the preceding claims, wherein the antibody molecule-target polypeptide docking model is constrained by adding one or more attractive constraints, wherein the attractive constraint is for a residue having an enrichment score greater than a first preselected value.

25. The method of claim 24, wherein the first preselected value is between 20% and 40%, e.g., between 25% and 35%, e.g., about 30%.

26. The method of claim 24 or 25, wherein the attractive constraint comprises a linearly scaled bonus based on the enrichment score.

27. The method of any of the preceding claims, wherein the antibody molecule-target polypeptide docking model is constrained by adding a repulsive constraint for a residue having an enrichment score less than a second preselected value.

28. The method of claim 27, wherein the second preselected value is between 5% and 20%, e.g., between 10% and 15%, e.g., about 12.5%.

29. The method of any of the preceding claims, wherein step (d) comprises generating a docked pose between a model of the antibody molecule and a model of the target polypeptide.

30. The method of any of the preceding claims, wherein step (d) comprises generating a plurality of docked poses between a model of the antibody molecule and a model of the target polypeptide.

31. The method of claim 30, wherein step (d) further comprises scoring the plurality of docked poses according to a docking algorithm, e.g., SnugDock.

32. The method of claim 31, wherein step (d) further comprises selecting a subset of the plurality of docked poses having the highest scores, e.g., the highest scoring 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000 or more docked poses.

33. The method of claim 32, wherein step (d) further comprises generating an ensemble docked pose using the selected subset of the plurality of docked poses, and setting the model of the antibody molecule and the model of the target polypeptide in accordance with the ensemble docked pose.

34. The method of any of claims 29-33, wherein the model of the antibody molecule comprises an ensemble antibody homology model derived from a plurality of homology models of the antibody.

35. The method of any of the preceding claims, wherein step (d) further comprises removing an antibody molecule-target polypeptide docketing model that exhibits a mode of engagement atypical for a known antibody-antigen complex, e.g., according to a structural filter derived from antibody-antigen crystal structure.

36. The method of any of the preceding claims, wherein step (d) comprises generating a plurality of antibody molecule-target polypeptide models.

37. The method of any of the preceding claims, wherein step (e) comprises identifying a plurality of sites on the target polypeptide that is capable of being bound by the antibody molecule.

38. A method of identifying an epitope on a target polypeptide, the method comprising:
(a) generating an antibody-target polypeptide docking model, wherein the antibody-target polypeptide docking model is constrained according to a plurality of enrichment scores determined by a method comprising:
(i) binding the antibody molecule to a plurality of variants of the target polypeptide,
(ii) obtaining (e.g., enriching) a plurality of variants exhibiting reduced binding to the antibody molecule, and
(iii) determining (e.g., calculating) enrichment scores for each of the plurality of the enriched variants; and
(b) identifying a site on the target polypeptide that is capable of being bound by the antibody molecule based on the antibody-target polypeptide docking model;
thereby identifying an epitope on a target polypeptide.

39. A method of identifying a paratope on an antibody molecule, the method comprising:
(a) binding the antibody molecule to a plurality of variants of the target polypeptide;
(b) obtaining (e.g., enriching) a plurality of variants exhibiting reduced binding to the antibody molecule;
(c) determining (e.g., calculating) enrichment scores for each of the plurality of the enriched variants;
(d) generating an antibody molecule-target polypeptide docking model, wherein the antibody-target polypeptide docking model is constrained according to the enrichment scores; and
(e) identifying one or more sites on the antibody molecule that is capable of being bound by the target polypeptide based on the antibody-target polypeptide docking model;
thereby identifying a paratope on an antibody molecule.

40. A method of identifying a paratope on an antibody, the method comprising:
(a) generating an antibody-target polypeptide docking model, wherein the antibody-target polypeptide docking model is constrained according to a plurality of enrichment scores determined (e.g., calculated) by a method comprising:
(i) binding the antibody to a plurality of variants of the target polypeptide,
(ii) obtaining (e.g., enriching) variants exhibiting reduced binding to the antibody molecule, and
(iii) determining (e.g., calculating) an enrichment score for each of the plurality of the obtained (e.g., enriched) variants; and
(b) identifying one or more sites on the antibody molecule that is capable of being bound by the target polypeptide based on the antibody-target polypeptide docking model;
thereby identifying a paratope on a target polypeptide.

41. An antibody molecule for which the epitope on a target polypeptide or the paratope on the antibody molecule for the target polypeptide is identified according to the method of any of the preceding claims.

42. A nucleic acid molecule encoding one or more chains (e.g., VH and/or VL) of the antibody molecule of claim 41.

43. A vector comprising the nucleic acid molecule of claim 42.

44. A host cell comprising the nucleic acid molecule of claim 42 or the vector of claim 43.

45. A method of making an antibody molecule, comprising culturing the host cell of claim 44 under conditions suitable for expression of the antibody molecule.
